(19) United States
(12) Patent Application Publication
(10) Pub. No.: US 2026/0207522 A1
(43) Pub. Date: Jul. 23, 2026

(54) YEAST CELL WALL PARTICLE ENCAPSULATION OF BIODEGRADABLE PRO-PAYLOADS

(71) Applicant: UNIVERSITY OF MASSACHUSETTS, Westborough, MA (US)

(72) Inventors: Gary R. OSTROFF, Worcester, MA (US); Ernesto R. SOTO, Worcester, MA (US)

(21) Appl. No.: 19/568,680

(22) Filed: Mar. 16, 2026

Related U.S. Application Data
(62) Division of application No. 16/981,072, filed on Mar. 15, 2019.
(60) Provisional application No. 62/644,219, filed on Mar. 16, 2018.

(51) Int. Cl. A61K 9/50; A61K 45/06

(52) U.S. Cl. CPC A61K 9/5068; A61K 45/06

(57) ABSTRACT

The present disclosure provides a particulate delivery system comprising an extracted yeast cell wall comprising beta-glucan and a pro-payload molecule comprising a payload scaffolding molecule operably and reversibly linked to a payload molecule through a cleavable linker. The disclosure further provides methods of making and methods of using the particulate delivery system.

### CROSS-REFERENCE TO RELATED APPLICATIONS

This application is a divisional of U.S. patent application Ser. No. 16/981,072, filed Sep. 15, 2020, which is a 35 U.S.C. § 371 filing of International Patent Application No. PCT/US2019/022408, filed Mar. 15, 2019, which claims priority to U.S. Provisional Patent Application No. 62/644,219, filed Mar. 16, 2018, the entire contents of which are incorporated herein by reference.

### STATEMENT OF FEDERALLY SPONSORED RESEARCH

This invention was made with government support under Grant Nos. NIFA 2015-11323 awarded by the United States Department of Agriculture (USDA) National Institutes of Food and Agriculture (NIFA). The government has certain rights in this invention.

### FIELD OF THE INVENTION

The present invention relates to medicine, pharmacology, and agriculture. More specifically, the invention relates to compositions and delivery systems for drug payloads.

### BACKGROUND

Glucan microparticles derived from Baker's yeast have been previously used to encapsulate soluble and small (<30 nm) nanoparticulate materials through the synthesis of polyplexes from soluble components, through Layer by Layer (LbL) adsorption of a soluble component(s) onto the surface of previously formed glucan particle encapsulated polyplexes, or through the adsorption of soluble component(s) onto the surface or within a cavity of pre-formed nanoparticles encapsulated in glucan particles. See PCT Patent Application Publications WO2005/0281781, WO2007/050643, and WO2012/024229; United States Patent Application Publications US2009/0209624, US2009/0226528, US2010/0221357, US2012/0070376, US2013/0065941, US2014/0335047, US2014/0350066, and US2016/0175251; and U.S. Pat. Nos. 7,740,861, 8,580,275, 9,662,299, and 9,682,135, the contents of which are incorporated herewith in their entirety.

Nevertheless, some water-soluble payloads cannot be trapped by any existing methods. Although these payloads can be loaded inside yeast cell wall particles (YCWPs), the water-soluble payloads rapidly diffuse out of the YCWPs. Several approaches have been attempted to improve payload retention by using polymer-based gels to plug seal the YCWP pores, and by co-loading of payloads with a hydrophobic lipid material to form a payload/lipid core that slows diffusion of the payload from the YCWP. However, these approaches have shown limited success to slow certain classes of payload diffusion from the YCWP.

In other instances, payload entrapment requires the use of potentially toxic heterobifunctional crosslinking agents or payloads with free amines, thereby resulting in a series of diverse chemical structures due to reductive amination chemistry. When employed, covalent linkages often hinder the release of payloads at their sites of delivery. Importantly, the amount of drug that can be encapsulated with these conventional methods is limited to approximately 2% of the glucan shell weight.

Thus, there is a need in the pharmaceutical and agricultural arts for the development of compositions and methods for delivering highly-diffusible, water-soluble payloads to cells and organisms.

### SUMMARY

In one aspect, the present disclosure provides a particulate delivery system comprising an extracted yeast cell wall comprising beta-glucan and a pro-payload molecule comprising a payload molecule operably linked to a payload scaffolding molecule through a chemical linker. The particulate delivery system of the present invention is useful for both in vivo and in vitro delivery of payload molecules to cells in a controlled manner.

In certain embodiments, the linker of the particulate delivery system is selected from the group consisting of an amide, an acetal, an anhydride, an aminocarboxylic acid, a carbamate, a cycloalkane, a disulfide, an enamine, an ester, a polyester, a hydrazide, a hydrazone, and urea.

In certain embodiments, linker of the particulate delivery system is cleavable by chemical or enzymatic hydrolysis. In certain embodiments, the linker is cleavable by pH-dependent hydrolysis. In certain embodiments, the linker is cleavable with a reagent selected from the group consisting of an enzyme, a reducing agent, an oxidizing agent, an acid, a base, and an organometallic or metal reagent. In certain embodiments, the enzyme is selected from the group consisting of a carboxylase, an esterase, and a urease.

In certain embodiments, the payload scaffolding molecule of the particulate delivery system comprises a chemical moiety selected from the group consisting of acetylacetone, anhydride, cyclohexane, cyclohexane 1,2,4, 5, tetracarboxylic acid, ethylenediaminetetraacetic acid (EDTA), isophorone diisocyanate, lauric acid, and poly(amidoamine).

In certain embodiments, the extracted yeast cell wall of the particulate delivery system comprises less than 90 weight percent beta-glucan. In certain embodiments, the extracted yeast cell wall comprises less than 30 weight percent chitin.

In certain embodiments, the payload molecule of the particulate delivery system comprises a reactive moiety selected from the group consisting of an amine, an aldehyde, a carbonyl, a carboxylic acid, a hydrazine, a hydroxyl, and a ketone. In certain embodiments, the payload molecule is selected from the group consisting of a polynucleotide, a peptide, a protein, a small organic active agent, a small inorganic active agent, and a mixture thereof.

In certain embodiments, the payload molecule of the particulate delivery system is selected from the group consisting of a microbicide, fungicide, insecticide, nematocide, a pesticide, an antibiotic, an analgesic, a non-steroidal anti-inflammatory drug (NSAID), a terpene, a terpenoid, a tetrahydrocannabinol, a cannabidiol, chemotherapeutic, a dietary supplement, and a mixture thereof.

In certain embodiments, the reactive moiety of the particulate delivery system is a hydroxyl. In certain embodiments, the payload molecule is selected from the group consisting of carvacrol, eugenol, geraniol, resveratrol, tetrahydrocannabinol, cannabidiol, acetaminophen, and curcumin.

In certain embodiments, the payload molecule is carvacrol, and the pro-payload molecule is dicarvacrol-EDTA.

In certain embodiments, the payload molecule is carvacrol, and the pro-payload molecule is dicarvacrol-cyclohexane.

In certain embodiments, the payload molecule is selected from the group consisting of geraniol, eugenol, thymol, and a combination thereof, and the pro-payload molecule is selected from the group consisting of di-geraniol-EDTA, di-eugenol-EDTA, di-thymol-EDTA, and a combination thereof.

In certain embodiments, the reactive moiety of the particulate delivery system is an amine. In certain embodiments, the payload molecule is selected from the group consisting of daunomycin, doxorubicin, cis-aconityl-doxorubicin, gentamicin, capreomycin, neomycin, and acetaminophen.

In certain embodiments, the payload molecule is doxorubicin, and the pro-payload molecule is poly(amidoamine)-doxorubicin.

In certain embodiments, the payload molecule is cis-aconityl-doxorubicin, and the pro-payload molecule is poly(amidoamine)-cis-aconityl-doxorubicin.

In certain embodiments, the reactive moiety is a carbonyl. In certain embodiments, the payload molecule is selected from the group consisting of doxorubicin, gentamicin, neomycin, cefoxitin, rifampicin, and camptothecin.

In certain embodiments, the payload molecule is doxorubicin, and the pro-payload molecule is doxorubicin-isoniazid.

In certain embodiments, the reactive moiety of the particulate delivery system is a carboxylic acid or a hydroxyl. In certain embodiments, the payload molecule is selected from the group consisting of ibuprofen, nicotinic acid, vancomycin, rifampicin, naproxen, ketoprofen, and betulinic acid or other carboxylic acid containing triterpenoids.

In certain embodiments, the payload molecule is naproxen, and the pro-payload molecule is naproxen-anhydride.

In certain embodiments, the reactive moiety of the particulate delivery system is selected from the group consisting of an amine and a hydroxyl. In certain embodiments, the payload molecule is selected from the group consisting of carvacrol and doxorubicin.

In certain embodiments, the payload molecule is doxorubicin, and the pro-payload molecule is doxorubicin-isophorone diisocyanate.

In certain embodiments, the reactive moiety of the particulate delivery system is selected from the group consisting of an amine, a ketone, and an aldehyde.

In certain embodiments, the payload molecule is cycloserine, and the pro-payload molecule is cycloserine-acetylacetone.

In certain embodiments, the reactive moiety is selected from the group consisting of an amine, a hydrazine, and a carbonyl.

In certain embodiments, the payload molecule is isoniazid, and the pro-payload molecule is isoniazid-lauric acid.

In another aspect, the present disclosure provides a kit comprising a first container containing a pro-payload molecule of the present disclosure comprising a payload scaffolding molecule operably and reversibly linked to a payload molecule through a cleavable linker, wherein the payload molecule is selected from the group consisting of a polynucleotide, a peptide, a protein, a small organic active agent, a small inorganic active agent, a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, a chemotherapeutic, a dietary supplement, and mixtures thereof; a second container containing a particulate delivery system comprising a yeast cell wall particle; and instructions for use.

In another aspect, the present disclosure provides a pharmaceutical composition comprising a particulate delivery system comprising a yeast cell wall particle, a pro-payload molecule of the present disclosure comprising a payload scaffolding molecule operably and reversibly linked to payload molecule through a cleavable linker, wherein the payload molecule is selected from the group consisting of a polynucleotide, a peptide, a protein, a small organic active agent, a small inorganic active agent, a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, a chemotherapeutic, a dietary supplement, and mixtures thereof; and a pharmaceutically acceptable excipient.

In another aspect, the present disclosure provides methods of using the particulate delivery system. In certain embodiments, the disclosure provides a method of delivering a payload molecule of the present disclosure to a cell, comprising: (a) reacting a payload molecule with a payload scaffolding molecule to form an insoluble pro-payload molecule, wherein the payload scaffolding molecule and the payload molecule are operably and reversibly linked through a cleavable linker; (b) contacting an extracted yeast cell wall with the pro-payload molecule, the extracted yeast cell wall defining an internal space and comprising beta glucan, wherein the pro-payload molecule becomes at least partially enclosed within the internal space, thereby forming a particulate delivery system; and (c) contacting a cell with the particulate delivery system under conditions that permit internalization of the particulate delivery system, cleavage of the cleavable linker, and release and delivery of the payload molecule within the cell.

In certain embodiments of the delivery method, the linker of the particulate delivery system is selected from the group consisting of an amide, an acetal, an anhydride, an aminocarboxylic acid, a carbamate, a cycloalkane, a disulfide, an enamine, an ester, a polyester, a hydrazide, a hydrazone, and urea.

In certain embodiments of the delivery method, the linker of the particulate delivery system is cleavable by chemical or enzymatic hydrolysis. In certain embodiments of the delivery method, the linker is cleavable by pH-dependent hydrolysis. In certain embodiments of the delivery method, the linker is cleavable with a reagent selected from the group consisting of an enzyme, a reducing agent, an oxidizing agent, an acid, a base, and an organometallic or metal reagent. In certain embodiments of the delivery method, the enzyme is selected from the group consisting of a carboxylase, an esterase, and a urease.

In certain embodiments of the delivery method, the payload scaffolding molecule of the particulate delivery system comprises a chemical moiety selected from the group consisting of acetylacetone, anhydride, cyclohexane, cyclohexane 1,2,4, 5, tetracarboxylic acid, ethylenediaminetetraacetic acid (EDTA), isophorone diisocyanate, lauric acid, and poly(amidoamine).

In certain embodiments of the delivery method, the extracted yeast cell wall of the particulate delivery system comprises less than 90 weight percent beta-glucan. In certain embodiments of the delivery method, the extracted yeast cell wall comprises less than 30 weight percent chitin.

In certain embodiments of the delivery method, the payload molecule of the particulate delivery system comprises a reactive moiety selected from the group consisting of an amine, an aldehyde, a carbonyl, a carboxylic acid, a hydrazine, a hydroxyl, and a ketone. In certain embodiments of the delivery method, the payload molecule is selected from the group consisting of a polynucleotide, a peptide, a protein, a small organic active agent, a small inorganic active agent, and a mixture thereof.

In certain embodiments of the delivery method, the payload molecule of the particulate delivery system is selected from the group consisting of a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, chemotherapeutic, a dietary supplement, and a mixture thereof.

In certain embodiments of the delivery method, the reactive moiety of the particulate delivery system is a hydroxyl. In certain embodiments of the delivery method, the payload molecule is selected from the group consisting of carvacrol, eugenol, geraniol, resveratrol, tetrahydrocannabinol, cannabidiol, acetaminophen, and curcumin.

In certain embodiments of the delivery method, the payload molecule is carvacrol, and the pro-payload molecule is dicarvacrol-EDTA.

In certain embodiments of the delivery method, the payload molecule is carvacrol, and the pro-payload molecule is dicarvacrol-cyclohexane.

In certain embodiments of the delivery method, the payload molecule is selected from the group consisting of geraniol, eugenol, thymol, and a combination thereof, and the pro-payload molecule is selected from the group consisting of di-geraniol-EDTA, di-eugenol-EDTA, di-thymol-EDTA, and a combination thereof.

In certain embodiments of the delivery method, the reactive moiety of the particulate delivery system is an amine. In certain embodiments of the delivery method, the payload molecule is selected from the group consisting of daunomycin, doxorubicin, cis-aconityl-doxorubicin, gentamicin, capreomycin, neomycin, and acetaminophen.

In certain embodiments of the delivery method, the payload molecule is doxorubicin, and the pro-payload molecule is poly(amidoamine)-doxorubicin.

In certain embodiments of the delivery method, the payload molecule is cis-aconityl-doxorubicin, and the pro-payload molecule is poly(amidoamine)-cis-aconityl-doxorubicin.

In certain embodiments of the delivery method, the reactive moiety is a carbonyl. In certain embodiments of the delivery method, the payload molecule is selected from the group consisting of doxorubicin, gentamicin, neomycin, cefoxitin, rifampicin, and camptothecin.

In certain embodiments of the delivery method, the payload molecule is doxorubicin, and the pro-payload molecule is doxorubicin-isoniazid.

In certain embodiments of the delivery method, the reactive moiety of the particulate delivery system is a carboxylic acid or a hydroxyl. In certain embodiments of the delivery method, the payload molecule is selected from the group consisting of ibuprofen, nicotinic acid, vancomycin, rifampicin, naproxen, ketoprofen, and betulinic acid or other carboxylic acid containing triterpenoids.

In certain embodiments of the delivery method, the payload molecule is naproxen, and the pro-payload molecule is naproxen-anhydride.

In certain embodiments of the delivery method, the reactive moiety of the particulate delivery system is selected from the group consisting of an amine and a hydroxyl. In certain embodiments of the delivery method, the payload molecule is selected from the group consisting of carvacrol and doxorubicin.

In certain embodiments of the delivery method, the payload molecule is doxorubicin, and the pro-payload molecule is doxorubicin-isophorone diisocyanate.

In certain embodiments of the delivery method, the reactive moiety of the particulate delivery system is selected from the group consisting of an amine, a ketone, and an aldehyde.

In certain embodiments of the delivery method, the payload molecule is cycloserine, and the pro-payload molecule is cycloserine-acetylacetone.

In certain embodiments of the delivery method, the reactive moiety is selected from the group consisting of an amine, a hydrazine, and a carbonyl.

In certain embodiments of the delivery method, the payload molecule is isoniazid, and the pro-payload molecule is isoniazid-lauric acid.

In another aspect, the present disclosure provides methods of making the particulate delivery system. In certain embodiments, the disclosure provides a method of making a particulate delivery system comprising: (a) reacting a payload molecule with a payload scaffolding molecule to form an insoluble pro-payload molecule, wherein the payload scaffolding molecule and the payload molecule are operably and reversibly linked through a cleavable linker; and (b) contacting an extracted yeast cell wall with the pro-payload molecule, the extracted yeast cell wall defining an internal space and comprising beta-glucan, wherein the pro-payload molecule becomes at least partially enclosed within the internal space, thereby forming the particulate delivery system.

In certain embodiments of the method of making, the linker of the particulate delivery system is selected from the group consisting of an amide, an acetal, an anhydride, an aminocarboxylic acid, a carbamate, a cycloalkane, a disulfide, an enamine, an ester, a polyester, a hydrazide, a hydrazone, and urea.

In certain embodiments of the method of making, the linker of the particulate delivery system is cleavable by chemical or enzymatic hydrolysis. In certain embodiments of the delivery method, the linker is cleavable by pH-dependent hydrolysis. In certain embodiments of the method of making, the linker is cleavable with a reagent selected from the group consisting of an enzyme, a reducing agent, an oxidizing agent, an acid, a base, and an organometallic or metal reagent. In certain embodiments of the method of making, the enzyme is selected from the group consisting of a carboxylase, an esterase, and a urease.

In certain embodiments of the method of making, the payload scaffolding molecule of the particulate delivery system comprises a chemical moiety selected from the group consisting of acetylacetone, anhydride, cyclohexane, cyclohexane 1,2,4, 5, tetracarboxylic acid, ethylenediaminetetraacetic acid (EDTA), isophorone diisocyanate, lauric acid, and poly(amidoamine).

In certain embodiments of the method of making, the extracted yeast cell wall of the particulate delivery system comprises less than 90 weight percent beta-glucan. In certain embodiments of the method of making, the extracted yeast cell wall comprises less than 30 weight percent chitin.

In certain embodiments of the method of making, the payload molecule of the particulate delivery system comprises a reactive moiety selected from the group consisting of an amine, an aldehyde, a carbonyl, a carboxylic acid, a hydrazine, a hydroxyl, and a ketone. In certain embodiments of the method of making, the payload molecule is selected from the group consisting of a polynucleotide, a peptide, a protein, a small organic active agent, a small inorganic active agent, and a mixture thereof.

In certain embodiments of the method of making, the payload molecule of the particulate delivery system is selected from the group consisting of a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, chemotherapeutic, a dietary supplement, and a mixture thereof.

In certain embodiments of the method of making, the reactive moiety of the particulate delivery system is a hydroxyl. In certain embodiments of the method of making, the payload molecule is selected from the group consisting of carvacrol, eugenol, geraniol, resveratrol, tetrahydrocannabinol, cannabidiol, acetaminophen, and curcumin.

In certain embodiments of the method of making, the payload molecule is carvacrol, and the pro-payload molecule is dicarvacrol-EDTA.

In certain embodiments of the method of making, the payload molecule is carvacrol, and the pro-payload molecule is dicarvacrol-cyclohexane.

In certain embodiments of the method of making, the payload molecule is selected from the group consisting of geraniol, eugenol, thymol, and a combination thereof, and the pro-payload molecule is selected from the group consisting of di-geraniol-EDTA, di-eugenol-EDTA, di-thymol-EDTA, and a combination thereof.

In certain embodiments of the method of making, the reactive moiety of the particulate delivery system is an amine. In certain embodiments of the method of making, the payload molecule is selected from the group consisting of daunomycin, doxorubicin, cis-aconityl-doxorubicin, gentamicin, capreomycin, neomycin, and acetaminophen.

In certain embodiments of the method of making, the payload molecule is doxorubicin, and the pro-payload molecule is poly(amidoamine)-doxorubicin.

In certain embodiments of the method of making, the payload molecule is cis-aconityl-doxorubicin, and the pro-payload molecule is poly(amidoamine)-cis-aconityl-doxorubicin.

In certain embodiments of the method of making, the reactive moiety is a carbonyl. In certain embodiments of the delivery method, the payload molecule is selected from the group consisting of doxorubicin, gentamicin, neomycin, cefoxitin, rifampicin, and camptothecin.

In certain embodiments of the method of making, the payload molecule is doxorubicin, and the pro-payload molecule is doxorubicin-isoniazid.

In certain embodiments of the method of making, the reactive moiety of the particulate delivery system is a carboxylic acid or a hydroxyl. In certain embodiments of the method of making, the payload molecule is selected from the group consisting of ibuprofen, nicotinic acid, vancomycin, rifampicin, naproxen, ketoprofen, and betulinic acid or other carboxylic acid containing triterpenoids.

In certain embodiments of the method of making, the payload molecule is naproxen, and the pro-payload molecule is naproxen-anhydride.

In certain embodiments of the method of making, the reactive moiety of the particulate delivery system is selected from the group consisting of an amine and a hydroxyl. In certain embodiments of the method of making, the payload molecule is selected from the group consisting of carvacrol and doxorubicin.

In certain embodiments of the method of making, the payload molecule is doxorubicin, and the pro-payload molecule is doxorubicin-isophorone diisocyanate.

In certain embodiments of the method of making, the reactive moiety of the particulate delivery system is selected from the group consisting of an amine, a ketone, and an aldehyde.

In certain embodiments of the method of making, the payload molecule is cycloserine, and the pro-payload molecule is cycloserine-acetylacetone.

In certain embodiments of the method of making, the reactive moiety is selected from the group consisting of an amine, a hydrazine, and a carbonyl.

In certain embodiments of the method of making, the payload molecule is isoniazid, and the pro-payload molecule is isoniazid-lauric acid.

The foregoing and other features and advantages of the disclosed drug delivery system and methods will be apparent from the following description of the embodiments of the system and method as illustrated in the accompanying drawings in which like reference characters refer to the same parts throughout the different views.

### BRIEF DESCRIPTION OF THE DRAWINGS

FIG. 1 is a schematic diagram depicting conventional methods of improving payload retention within yeast cell wall particles.

FIGS. 2A-2B are schematic diagrams depicting the claimed method of creating water-insoluble pro-payload molecules (FIG. 2A) for encapsulation within yeast cell wall particles (FIG. 2B).

FIG. 3 is a schematic diagram depicting the creation of a pro-payload molecule containing an ester biodegradable linker from payloads containing hydroxyl groups.

FIG. 4 is a schematic diagram depicting examples of dicarvacrol-EDTA and dicarvacrol-cyclohexane pro-payload molecules containing biodegradable aminocarboxylic acid linkages.

FIG. 5 is a schematic diagram depicting the creation of the dicarvacrol-EDTA pro-payload molecule containing a biodegradable linker. The ester linkages (yellow triangles) may be hydrolyzed by enzymatic cleavage, e.g., with an esterase, thereby releasing the two carvacrol payload molecules (green rectangles) from the EDTA backbone (red oval).

FIG. 6 is a line graph, micrograph, and table, depicting the results of an in vitro carvacrol release assay, all of which show that carvacrol release is delayed in encapsulated dicarvacrol-EDTA pro-payload molecules (YP-Dicarvacrol EDTA) compared to encapsulated non-pro-payload molecules (YP-Carvacrol).

FIG. 7 is a schematic diagram depicting an in vitro bacterial inhibition assay, and a bar graph showing that YP-dicarvacrol-EDTA retained antimicrobial activity against E. coli in the assay.

FIG. 8A is a schematic diagram depicting a payload release assay. FIG. 8B is a bar graph depicting that the chemical linkage in YP-dicarvacrol-EDTA (grey bar) is resistant to digestion with gastric fluids (SGF/pepsin) but susceptible to digestion with intestinal fluids (SIF/pancreatin). FIG. 8C is a line graph depicting the release of YP-dicarvacrol-EDTA over time following the addition of SIF/pancreatin.

FIG. 9 is a schematic diagram depicting a microbial inhibition assay, and a table showing that antimicrobial activity was localized to the pelleted fraction of YP-dicarvacrol-EDTA, indicating that YP-dicarvacrol-EDTA is resistant to linker hydrolysis during simulated digestion, whereas the YP-carvacrol control readily released the payload after treatment with simulated gastric and intestinal fluids.

FIG. 10 is a bar graph showing that YP-carvacrol-EDTA and YP-carvacrol were equally effective against an intestinal parasitic worm (Cayathostomin) in an in vitro egg to larvae assay.

FIG. 11 is a schematic diagram depicting an ultraviolent (UV) exposure experiment, and a bar graph showing that the stability of YP-dicarvacrol-EDTA was not affected by UV light radiation.

FIG. 12 is a schematic diagram depicting a method of synthesizing encapsulated dicarvacrol-EDTA in situ. The results, shown in the bar graph and micrograph, demonstrate it is not possible to synthesize dicarvacrol-EDTA in situ in high yield compared to the efficient synthesis method depicted in FIGS. 2A-2B.

FIG. 13 is a schematic diagram depicting the creation of the dicarvacrol-cyclohexane (DCC6) pro-payload molecule.

FIG. 14 is a schematic diagram depicting the loading of pro-payload dicarvacrol-cyclohexane (DCC6) into extracted yeast cell wall particles in a 1:1 weight ratio to create encapsulated DCC6, and micrograph showing that DCC6 was successfully encapsulated.

FIGS. 15A-15B are line graphs and tables depicting the results of a carvacrol release assay and an in vitro bacterial inhibition assay. FIG. 15A is a line graph and 15B is a table that show that carvacrol release is delayed in encapsulated dicarvacrol-EDTA (YP-dicarvacrol-EDTA) and dicarvacrol-cyclohexane (YP-DCC6) pro-payload molecules. FIG. 15C is a table showing that YP-dicarvacrol-EDTA, YP-DCC6, and YP-carvacrol were all similarly effective in the in vitro bacterial inhibition assay.

FIG. 16 is a schematic diagram depicting the creation of pro-payload di-terpene-EDTA pro-payload molecules synthesized by reacting the payload molecules geraniol, eugenol, and thymol with the payload scaffolding molecule EDTA.

FIG. 17 is a schematic diagram depicting the creation of encapsulated di-terpene-EDTA pro-payload mixtures (YP-d(GET) EDTA).

FIG. 18 are a series of line graphs and tables depicting the results of an in vitro release assay, showing that encapsulated pro-payloads YP-Geraniol EDTA, YP-Eugenol EDTA, and YP-Thymol EDTA all demonstrated improved diffusion kinetics compared to a non-pro-payload mixture of geraniol, eugenol, and thymol.

FIG. 19 is a line graph, micrograph, and table, depicting the results of an in vitro release assay, which shows that encapsulated pro-payloads of the combined terpenes demonstrated improved diffusion kinetics compared to a non-pro-payload mixture of geraniol, eugenol, and thymol.

FIG. 20 is a bar graph depicting the results of an antifungal activity assay, showing that the encapsulated mixture of terpene pro-payloads (i.e., YP-d(GET) EDTA 424), as well as the encapsulated pro-payload geraniol (i.e., YP-dG EDTA), demonstrated antifungal activity against the yeast S. cerevisae.

FIG. 21 is a schematic diagram depicting the creation of pro-payload molecules containing carbamate or urea biodegradable linkers from payloads containing hydroxyl or amine groups.

FIG. 22 is a schematic diagram depicting the creation of the pro-payload molecule doxorubicin-isophorone diisocyanate (Dox-IPDI) by reacting doxorubicin (Dox) with the scaffolding payload molecule isophorone diisocyanate (IPDI).

FIG. 23 is a schematic diagram, a micrograph, and a line graph, all of which show that the Dox-IPDI pro-drug is loaded into YCWPs more efficiently than Dox alone.

FIG. 24 is a table demonstrating that the release of the Dox-IPDI pro-drug is controlled by pH-dependent cleavage of the pro-drug linker.

FIG. 25 is a schematic diagram depicting an in vitro macrophage delivery assay, and a line graph which demonstrates that YCWP-Dox-IPDI delivered doxorubicin more efficiently to macrophages than either YCWP-Dox or the soluble Dox control.

FIG. 26 is a schematic diagram depicting the creation of a pro-payload molecule containing hydrazone biodegradable linker from payloads containing carbonyl groups.

FIG. 27 is a schematic diagram depicting the creation of the pro-payload molecule doxorubicin-isoniazid (Dox-INH) by reacting doxorubicin (Dox) with the scaffolding payload molecule isonicotinylhydrazide (INH).

FIG. 28 schematic diagram depicting the loading of the Dox-INH prodrug into YCWPs, and a micrograph and table demonstrating that the Dox-INH pro-drug is loaded into YCWPs more efficiently than Dox alone.

FIG. 29 a table demonstrating that the release of the Dox-INH pro-drug is controlled by pH-dependent cleavage of the pro-drug linker.

FIG. 30 is a schematic diagram of an in vitro macrophage delivery assay, and a line graph which demonstrates that YCWP-Dox-INH delivered doxorubicin more efficiently to macrophages than either YCWP-Dox or the soluble Dox control.

FIG. 31 is a schematic diagram depicting the creation of a pro-payload molecule containing an amide biodegradable linker from payloads containing amine groups.

FIG. 32 is a schematic diagram depicting the creation of the pro-payload molecule PAMAM-doxorubicin (PAMAM-Dox) by reacting doxorubicin (Dox) with the scaffolding payload molecule PAMAM generation 5 (G 5.0).

FIG. 33 is a schematic diagram depicting the creation of the pro-payload molecule PAMAM-cis-aconitic-doxorubicin (PAMAM-CAD) by reacting doxorubicin with the scaffolding payload molecule PAMAM generation 5 (G 5.0) and cis-aconitic anhydride.

FIG. 34 is a schematic diagram depicting the loading of PAMAM-Dox and PAMAM-CAD pro-drugs into YCWPs, and a micrograph and table demonstrating that PAMAM-Dox and PAMAM-CAD pro-drugs are loaded into YCWPs more efficiently than Dox alone.

FIG. 35 is a table demonstrating that the release of the PAMAM-Dox and PAMAM-CAD pro-drugs are controlled by pH-dependent cleavage of the pro-drug linkers, and that PAMAM-CAD contains a more acid labile linker than PAMAM-Dox.

FIG. 36 is a schematic diagram depicting an in vitro macrophage delivery assay, and a line graph which demonstrates that YCWP-PAMAM-CAD delivered doxorubicin more efficiently to macrophages than YCWP-PAMAM-Dox, YCWP-Dox, or the soluble Dox control.

FIG. 37 is a schematic diagram depicting the creation of a pro-payload molecule containing anhydride and ester biodegradable linkers from payloads containing hydroxyl and carboxylic groups.

FIG. 38 is a schematic diagram depicting the creation of the pro-payload molecule naproxen anhydride (Nap-An) by reacting naproxen (Nap) with ethanoic anhydride (An).

FIG. 39 is a table demonstrating that the release of the Nap-An pro-drug is controlled by pH-dependent cleavage of the pro-drug linker.

FIG. 40 is a schematic diagram depicting an in vitro macrophage delivery assay, and a table which demonstrates that YCWP-Nap-An delivered naproxen more efficiently to macrophages and inhibited TNF-alpha more than either YCWP-Nap or the free Nap control.

FIG. 41 schematic diagram depicting the creation of the pro-payload molecule cycloserine acetylacetone (CS-AcA) by reacting cycloserine with the scaffolding payload molecule acetylacetone.

FIG. 42 is a schematic diagram depicting the loading of the CS-AcA pro-drug into YCWPs, and a table demonstrating that the CS-AcA pro-drug is loaded into YCWPs more efficiently than cycloserine alone.

FIG. 43 is a table demonstrating that YCWP-CS-AcA retained the ability to inhibit the survival of the bacterium Staphylococcus aureus in an in vitro antimicrobial assay.

FIG. 44 is a schematic diagram depicting the creation of the pro-payload molecule isoniazid lauric acid (INH-LA) by reacting isoniazid (INH) with the scaffolding payload molecule lauric acid (LA).

FIG. 45 is a schematic diagram depicting the loading of the INH-LA pro-drug into YCWPs, and a micrograph and table demonstrating that the INH-LA pro-drug is loaded into YCWPs more efficiently than INH alone.

### DETAILED DESCRIPTION

The present disclosure improves upon conventional encapsulation technologies by providing a particulate delivery system comprising an extracted yeast cell wall comprising beta-glucan and a pro-payload molecule comprising a payload molecule operably linked to a payload scaffolding molecule through a chemical linker. The term “operably linked” refers to a juxtaposition wherein the components so described are in a relationship permitting them to function in their intended manner.

In the present disclosure, payload molecules are chemically enjoined to payload scaffolding molecules in such a manner that the chemical or biologic activities of the payloads are not permanently altered or diminished. The methods of the present disclosure can achieve a loading capacity of greater than 50%, thereby, providing for a significant improvement over existing technologies.

The disclosures of patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

That the disclosure may be more readily understood, select terms are defined below.

### Payload Molecules

The particulate delivery system of the present disclosure is useful for in vivo or in vitro delivery of payload molecules to a cell or organism. This delivery system is useful for the delivery of highly-diffusible, water-soluble molecular payloads that cannot be trapped within encapsulating yeast cell well particles using any art-known method. Any molecular payload that can be modified to yield a water-insoluble payload derivative, i.e., a “pro-payload” is envisioned by the present disclosure. Payloads containing a chemical reactive group or moiety that can be reacted with a molecular backbone, i.e., a “payload scaffolding molecule,” to form a chemical bond that is susceptible to chemical (e.g., pH) and/or biological (e.g., enzyme) hydrolysis are envisioned and exemplified throughout the present application. In certain embodiments, payloads of the present disclosure contain one or more modifiable reactive moieties selected from the group consisting an amide, an aldehyde, a carbonyl, a carboxylic acid, a hydrazine, a hydroxyl, and a ketone.

In certain embodiments, the particulate delivery system of the present disclosure is useful for in vivo or in vitro delivery of payload molecules including, but limited to, polynucleotides such as oligonucleotides, antisense constructs, siRNA, enzymatic RNA, and recombinant DNA constructs, including expression vectors.

In other embodiments, the particulate delivery system of the present disclosure is useful for in vivo or in vitro delivery of payload molecules such as amino acids, peptides and proteins. By “protein” is meant a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15 kD, or at least about 20 kD.

The protein payload molecules are essentially pure and essentially homogeneous (i.e., free from contaminating proteins, etc.). “Essentially pure” protein, as used herein, means a composition comprising at least about 90% by weight of the protein, based on total weight of the composition, or at least about 95% by weight. “Essentially homogeneous” protein, as used herein, means a composition comprising at least about 99% by weight of protein, based on total weight of the composition. Proteins may be derived from naturally occurring sources or produced by recombinant technology. Proteins include protein variants produced by amino acid substitutions or by directed protein evolution (Kurtzman, A.L., et al., Advances in Directed Protein Evolution by Recursive Genetic Recombination: Applications to Therapeutic Proteins, Curr. Opin. Biotechnol. 2001 12(4): 361-70) as well as derivatives, such as PEGylated proteins.

In addition to peptides, polypeptides, and polynucleotides, the particulate delivery system of the present disclosure is suitable for the delivery of smaller molecules, e.g., for the delivery of small pharmaceutically active agents. Examples of agents that can be incorporated into the delivery system of the present disclosure include, without limitation, small inorganic active agents such as, but not limited to, aluminum hydroxide, calcium carbonate, magnesium carbonate, and sodium carbonate; narcotics such as, but not limited to, codeine, dihydrocodeine, meperidine, and morphine; non-narcotic analgesics and anti-inflammatory compounds, such as, but not limited to, salicylates, aspirin, acetaminophen, d-propoxyphene, fenoprofen, flurbiprofen, ibuprofen, ketoprofen, naproxen, and, oxaprozin; small active agents with antibiotic, antimicrobial, antifungal, antiparasitic, pesticidal, or insecticidal activities, such as, but not limited to, cycloserine, daunomycin, doxorubicin, gentamicin, capreomycin, isoniazid, neomycin, vancomycin, and rifampicin; chemotherapeutics such as, but not limited to, doxorubicin, epirubicin, daunorubicin, idarubicin, and mitoxantrone; and naturally-occurring organic active compounds for medicinal or nutritional use in an animals or humans, such as, but not limited to, carvacrol, eugenol, geraniol, resveratrol, thymol, curcumin, tetrahydrocannabinol, cannabidiol, terpenes, terpenoids, betulinic acid, and other carboxylic acid-containing triterpenoids.

### Pro-Payload Molecules

As used herein, the term “pro-payload” or “pro-payload molecule” refers to any payload molecule, as described herein or known to one of skill in the art, which has been chemically modified to yield a water-insoluble payload derivative. Pro-payload molecules are produced by chemically bonding a payload molecule to a payload scaffolding molecule or molecular backbone. The bond between the payload and scaffolding molecule constitutes a “linker” or “linkage” that is susceptible to chemical and/or biological hydrolysis that regenerates and releases the water-soluble payload.

In certain embodiments, a linker of the present disclosure is selected from the group consisting of an amide, an acetal, an anhydride, an aminocarboxylic acid, a carbamate, a cycloalkane, a disulfide, an enamine, an ester, a polyester, a hydrazide, a hydrazone, and urea.

In certain embodiments, the linker is cleavable by pH-dependent hydrolysis. In other embodiments, the linker is cleavable with a reagent selected from the group consisting of an enzyme, a reducing agent, an oxidizing agent, an acid, a base, and an organometallic or metal reagent. In other embodiments, the linker of is cleavable with an enzyme is selected from the group consisting of a carboxylase, an esterase, and a urease.

In certain embodiments, the payload scaffolding molecule of the particulate delivery system of the present disclosure comprises a chemical moiety selected from the group consisting of acetylacetone, anhydride, cyclohexane, cyclohexane 1,2,4, 5, tetracarboxylic acid, ethylenediaminetetraacetic acid (EDTA), isophorone diisocyanate, lauric acid, and poly(amidoamine).

In certain exemplary embodiments, the present disclosure provides compositions and methods for the delivery of various therapeutics by yeast cell wall particles. These include, but are not limited to, a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, a chemotherapeutic, a dietary supplement, and mixtures thereof

### A. Terpene Pro-Payloads

In certain embodiments, the disclosure provides compositions and methods for the encapsulation and delivery of terpene pro-payload molecules. Any terpene may be converted to a pro-payload molecule, encapsulated, and delivered according to the methods of the present disclosure. The terpene pro-payload component may comprise a single terpene or a mixture of terpenes.

The term “terpene” as used herein refers to terpenes of formula (C₅H₈)ₙ, and terpene derivatives, such as terpene aldehydes. In addition, reference to a single name of a compound will encompass the various isomers of that compound. For example, the term citral includes the cis-isomer citral-a (or geranial) and the trans-isomer citral-b (or neral).

Terpenes are classified as Generally Recognized as Safe (GRAS) and have been used for many years in the flavoring and aroma industries. The list of terpenes which are exempted from US regulations found in EPA regulation 40 C.F.R. Part 152 is incorporated herein by reference in its entirety. Terpenes have a relatively short life span of approximately 28 days once exposed to oxygen (e.g., air). Terpenes decompose to CO₂, further demonstrating the safety and environmental friendliness of the compositions and methods of the disclosure.

Terpenes have been found to inhibit the in vitro growth of bacteria and fungi (Chaumont et al.), Ann. Pharm. Fr., 1992, 50(3): 156-166; Moleyar et al., Int. J. Food Microbiol., 1992, 16(4): 337-342; and Pattnaik et al., Microbios., 1997, 89(358): 39-46) and some internal and external parasites (Hooser et al., J. Am. Vet. Med. Assoc., 1986, 189(8): 905-908). The terpene geraniol is the active component (75%) of rose oil. Rose oil and geraniol at a concentration of 2 mg/L inhibited the in vitro growth of H. pylori. Geraniol was found to inhibit the growth of C. albicans and S. cerevisiae strains by enhancing the rate of potassium leakage and disrupting membrane fluidity (Bard et al., Lipids, 1998, 23(6): 534-538).

There may be different modes of action of terpenes against microorganisms; they (1) interfere with the phospholipid bilayer of the cell membrane, (2) impair a variety of enzyme systems (HMG-reductase), and (3) destroy or inactivate genetic material. It is believed that due to the modes of action of terpenes being so basic, e.g., blocking of cholesterol, that infective agents do not build a resistance to terpenes.

The terpenes, surfactants, and other components of the pre-payloads according to the disclosure may be readily purchased or synthesized using techniques generally known to synthetic chemists. Useful terpenes according to the present disclosure, for safety and regulatory reasons, are at least food grade terpenes, as defined by the United States FDA or equivalent national regulatory body outside the USA. Non-limiting examples of suitable surfactants include sodium lauryl sulphate, polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, polyoxyethylenesorbitan, monooleate, Tw®, Span® 20, Span® 40, Span® 60, Span® 80, Brig 30 or mixtures thereof.

Alternatively, stable terpene solutions can be obtained by mixing terpenes and water at high shear. See PCT Patent Application Publication WO2003/020024. Regardless of how they are prepared, terpenes are prone to oxidation in aqueous emulsion systems, which make long term storage a problem. Thus, the composition of the present disclosure can comprise an antioxidant to reduce oxidation of the terpene. A non-limiting example of such an anti-oxidant might be rosemary oil, vitamin C, or vitamin E. Preservatives and other additives can also be present such as, for example, antimicrobials, anti-oxidants, chelating agents, and inert gases.

Terpenes can be taken up and stably encapsulated within hollow glucan particles or cell wall particles. See U.S. Pat. No. 9,439,416, the contents of which are incorporated by reference in its entirety. Encapsulation of terpenes into such particles can be achieved by incubation of the particles with the terpene. Nevertheless, terpenes rapidly diffuse from the glucan shell when encapsulated according to conventional methods. Accordingly, certain exemplary embodiments of the present disclosure provide for improved compositions and methods for the encapsulation and delivery of terpenes as controlled-release terpene pro-payloads with reduced diffusion.

Compositions of the present disclosure can comprise other active compounds, alone or in addition to the terpene component, for example, enzymes, or the like. The compositions can comprise a further active agent in addition to the terpene component, for example, an antimicrobial agent, an anti-fungal agent, an insecticidal agent, an anti-inflammatory agent, an anesthetic, or the like.

Suitable agents include, but are not limited to, antifungals, such as cell wall hydrolyases, to the extent they do not degrade the hollow glucan particle or cell wall particle, cell wall synthesis inhibitors, and standard antifungals; antibacterials, such as antiseptics, cell wall hydrolases, synthesis inhibitors, and antibiotics; and insecticides, such as natural insecticides and chitinase.

In certain embodiments of the present disclosure, the terpene pro-payload molecule is dicarvacrol-EDTA. In certain embodiments, the pro-payload molecule is dicarvacrol-cyclohexane. In certain embodiments, the pro-payload molecule is digeraniol-EDTA. In certain embodiments, the pro-payload molecule is dieugenol-EDTA. In certain embodiments, the pro-payload molecule is dithymol-EDTA.

### B. Antimicrobial Pro-Payloads

Certain exemplary embodiments of the present disclosure provide for compositions and methods for the encapsulation and delivery of pro-payload molecules with antimicrobial activity effective against classes of organisms such as Gram positive bacteria, Gram negative bacteria, fungi, and viruses.

As used herein, the term “antimicrobial” refers to the ability of a compound to inhibit or irreversibly prevent the growth of a microorganism. Such inhibition or prevention can be through a microbicidal action or microbistatic inhibition. The term “microbicidal inhibition” refers to the ability of the antimicrobial compound to kill, or irrevocably damage the target organism. The term “microbistatic inhibition” as used herein refers to the ability of the antimicrobial compound to inhibit the growth of the target organism without death.

A compound with microbicidal or microbistatic inhibitory properties can be applied to an environment either presently exhibiting microbial growth (i.e., therapeutic treatment) or to an environment at risk of supporting such growth (i.e., prevention or prophylaxis). An environment capable of sustaining microbial growth refers to a fluid, substance, or organism where microbial growth can occur or where microbes can exist. Such environments can be, for example, animal tissue or bodily fluids, water and other liquids, food, food products or food extracts, crops, and certain inanimate objects. It is not necessary that the environment promote the growth of the microbe, only that it permit its subsistence.

Any suitable antimicrobial compound may be incorporated into an antimicrobial pro-payload and encapsulated according to the methods presently described. In certain nonlimiting embodiments, the antimicrobial compound is an antibiotic, such as cycloserine, daunomycin, doxorubicin, gentamicin, capreomycin, isoniazid, neomycin, vancomycin, and rifampicin. For example, the pro-payload can be isoniazid-lauric acid, doxorubicin-isoniazid, or cycloserine-acetylacetone. The antimicrobial pro-payload component may comprise a single microbial or a mixture of antimicrobials.

### C. Chemotherapeutic Pro-Payloads

Certain exemplary embodiments of the present disclosure also provide compositions and methods for the encapsulation and delivery of pro-payload molecules with chemotherapeutic or anticancer properties. Any solid or hematological cancer may be treated with the pro-payload molecules presently disclosed.

Exemplary useful chemotherapeutic agents include alkylating agents, anti-metabolites, alkaloids, and miscellaneous agents (including hormones), and certain antibiotics. For example, anthracyclines are one of the more commonly used chemotherapeutic antibiotics. Anthracycline antibiotics are produced by the fungus Streptomyces peuceitius var. caesius. Anthracycline antibiotics have tetracycline ring structures with an unusual sugar, daunosamine, attached by glycosidic linkage. Cytotoxic agents of this class all have quinone and hydroquinone moieties on adjacent rings that permit them to function as electron-accepting and donating agents.

Anthracyclines achieve their cytotoxic effect by several mechanisms, including intercalation between DNA strands, thereby interfering with DNA and RNA synthesis; production of free radicals that react with and damage intracellular proteins and nucleic acids; chelation of divalent cations; and reaction with cell membranes. The wide range of potential sites of action may account for the broad efficacy as well as the toxicity of the anthracyclines.

Any suitable chemotherapeutic or antitumor compound may be incorporated into a pro-payload and encapsulated according to the methods presently described. In certain embodiments, the chemotherapeutic or antitumor compound is selected from the group consisting of doxorubicin, epirubicin, daunorubicin, idarubicin, and mitoxantrone. The chemotherapeutic or anticancer pro-payload component may comprise a single pro-payload molecule or a mixture of pro-payload molecules.

Doxorubicin (Dox) is one exemplary useful anthracycline that displays broader activity against human neoplasms, including a variety of solid tumors. In some non-limiting examples, the pro-payload is doxorubicin-isoniazid, doxorubicin-isophorone diisocyanate, poly(amidoamine)-doxorubicin, or poly(amidoamine)-cis-aconityl-doxorubicin.

### D. Non-Steroidal Anti-Inflammatory Drug (NSAID) Pro-Payloads

The disclosure also provides compositions and methods for the encapsulation and delivery of pro-payload molecules with analgesic and anti-inflammatory properties. The analgesic or anti-inflammatory pro-payload component may comprise a single pro-payload molecule or a mixture of pro-payload molecules. Any useful analgesic or anti-inflammatory compound may be incorporated into a pro-payload and encapsulated according to the methods presently described.

In certain embodiments, the analgesic or anti-inflammatory compound is selected from the group consisting of salicylates, aspirin, acetaminophen, d-propoxyphene, fenoprofen, flurbiprofen, ibuprofen, ketoprofen, naproxen, and oxaprozin.

Nonsteroidal anti-inflammatory drugs (NSAIDs) are a drug class that reduce pain, decrease fever, prevent blood clots and, in higher doses, decrease inflammation. Useful NSAIDs include, without limitation, aspirin, ibuprofen and naproxen.

Naproxen is a well-known NSAID, with a daily dose ranging from about 250 to about 1500 milligrams, or from about 500 to about 1000 milligrams. Naproxen, and other analgesic drugs, can be administered in multiple doses over 12 or 24 hours.

Additionally, a higher initial dose, followed by relatively low maintenance doses, can be delivered. See, e.g., Palmisano et al., Advances in Therapy, Vol. 5, No. 4, July/August 1988; describing the use of multiple doses of ketoprofen (initial dose of 150 mg followed by subsequent doses of 75 mg) and ibuprofen (initial dose of 800 mg followed by subsequent doses of 400 mg).

Controlled release pharmaceutical dosage forms can be used to optimize drug delivery and enhance patient compliance. A pharmaceutical dosage form can deliver more than one drug, each at a modified rate.

In certain non-limiting embodiments, the pro-payload molecule is naproxen-anhydride.

### Yeast Cell Wall Particles

The water-insoluble pro-payloads of the present disclosure may be dissolved in any solvent that is compatible with yeast cell wall glucan particles, e.g., dimethylsulfoxide (DMSO), ethanol, etc. After loading the pro-payloads into glucan shells, the glucan particle pro-payloads are processed to remove the solvent. The disclosed pro-payload technology offers improved payload stability, e.g., pro-payloads are water-insoluble and have slow hydrolysis at neutral pH.

Extracted “yeast cell wall particles” or “YCPs” are readily available, biodegradable, substantially spherical particles about 2-4 μm in diameter. Preparation of extracted yeast cell wall particles is known in the art, and is described, for example in U.S. Pat. Nos. 4,992,540, 5,082,936, 5,028,703, 5,032,401, 5,322,841, 5,401,727, 5,504,079, 5,968,811, 6,444,448, 6,476,003, published U.S. applications 2003/0216346 A1, 2004/0014715 A1, and PCT published application WO 02/12348 A2, which are specifically incorporated herein by reference.

In certain embodiments, the extracted yeast cell wall of the particulate delivery system of the present disclosure comprises less than 90 weight percent beta-glucan. In certain embodiments, the extracted yeast cell wall of the particulate delivery system of the present disclosure comprises less than 30 weight percent chitin.

### Articles of Manufacture, Compositions, and Methods

In another aspect, the present disclosure provides an article of manufacture or kit comprising a first container containing a pro-payload molecule comprising a payload scaffolding molecule operably and reversibly linked to a payload molecule through a cleavable linker, wherein the payload molecule is selected from the group consisting of a polynucleotide, a peptide, a protein, a small organic active agent, a small inorganic active agent, a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, a chemotherapeutic, a dietary supplement, and mixtures thereof, a second container containing a particulate delivery system comprising a yeast cell wall particle, and instructions for use.

In another aspect, the present disclosure provides methods of making a particulate delivery system comprising the steps of providing an extracted yeast cell wall comprising beta-glucan, the yeast cell wall defining an internal space; reacting a payload molecule with a payload scaffolding molecule to form an insoluble pro-payload molecule, wherein the payload scaffolding molecule and the payload molecule are operably and reversibly linked through a cleavable linker; and contacting the extracted yeast cell wall with the pro-payload molecule, wherein the pro-payload molecule becomes enclosed within the internal space, thereby forming the particulate delivery system.

In another aspect, the present disclosure provides a pharmaceutical composition comprising a particulate delivery system comprising a yeast cell wall particle, a pro-payload molecule comprising a payload scaffolding molecule operably and reversibly linked to payload molecule through a cleavable linker, wherein the payload molecule is selected from the group consisting of a polynucleotide, a polynucleotide, a peptide, a protein, a small organic active agent, a small inorganic active agent, a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, a chemotherapeutic, a dietary supplement, and mixtures thereof, and a pharmaceutically acceptable excipient.

In another aspect, the present disclosure provides methods of using the particulate delivery system. In certain embodiments, the disclosure provides a method of delivering a payload molecule of the present disclosure to a cell, comprising: (a) reacting a payload molecule with a payload scaffolding molecule to form an insoluble pro-payload molecule, wherein the payload scaffolding molecule and the payload molecule are operably and reversibly linked through a cleavable linker; (b) contacting an extracted yeast cell wall with the pro-payload molecule, the extracted yeast cell wall defining an internal space and comprising beta glucan, wherein the pro-payload molecule becomes at least partially enclosed within the internal space, thereby forming a particulate delivery system; and (c) contacting a cell with the particulate delivery system under conditions that permit internalization of the particulate delivery system, cleavage of the cleavable linker, and release and delivery of the payload molecule within the cell.

### Agricultural and Industrial Compositions and Methods

The compositions and methods of the present disclosure are useful in the fields of consumer and industrial products, e.g., in food, human and animal drugs, cosmetics, and agriculture. In some embodiments, the compositions and methods of the present disclosure extend to agricultural applications. In certain embodiments, the present disclosure relates to the development and delivery of stable and controlled-release microbiocides, fungicides, insecticides, nematocides, and pesticides to agricultural species, e.g., plants and/or animals.

Some embodiments of the present disclosure provide compositions and methods useful in the control of a variety of agricultural pests. As used herein, the term “pest” refers to organisms that negatively affect a host—such as a plant or an animal such as a mammal—by colonizing, damaging, attacking, competing with them for nutrients, or infecting them. This includes, e.g., microbes, fungi, weeds, nematodes, and arthropods. Arthropods include insects and arachnids, as well as sucking and biting pests such as mites, ticks, ants, and lice.

Certain embodiments of the present disclosure provide compositions and methods for use in controlling sucking and biting pests, including e.g., mosquitoes, ticks, lice, fleas, mites, flies, and spiders.

Certain embodiments of the present disclosure provide for compositions and methods for use in controlling nematodes. Nematodes (Kingdom: Animalia; Phylum:
Nematoda) are microscopic round worms. They can generally be described as aquatic, triploblastic, unsegmented, bilaterally symmetrical roundworms, that are colorless, transparent, usually bisexual, and worm-shaped (vermiform), although some can become swollen (pyroform).

Many nematodes are obligate parasites and a number of species constitute a significant problem in agriculture. Thus, methods to control their parasitic activities are an important feature in maximizing crop production in modern intensive agriculture.

Nematodes are not just parasitic to plants but a number of species are parasitic to animals, both vertebrate and invertebrate. Around 50 species attack humans and these include Hookworm (Anclyostoma), Strongylids (Strongylus), Pinworm (Enterolobius), Trichinosis (Trichina), Elephantitis (Wuchereria), Heartworm (Dirofilaria), and Ascarids (Ascaris).

In some embodiments of the present disclosure, any of the compositions described above may be formulated in a deliverable form suited to a particular application. Deliverable forms that can be used in accordance with embodiments of the present disclosure include, but are not limited to, liquids, emulsions, emulsifiable concentrates, solids, aqueous suspensions, oily dispersions, pastes, granules, powders, dusts, fumigants, and aerosol sprays. Suitable deliverable forms can be selected and formulated by those skilled in the art using methods currently known in the art. The compositions can be provided in combination with an agriculturally, food, or pharmaceutically acceptable carrier or excipient in a liquid, solid, or gel-like form. For solid compositions, suitable carriers include pharmaceutical or food grades of mannitol, lactose, starch, magnesium stearate, sodium saccharin, talc, cellulose, glucose, sucrose, and magnesium carbonate. Suitably, the formulation is in tablet or pellet form. As suitable carrier could also be a human or animal food material. Additionally, conventional agricultural carriers could also be used.

The use of terpenes to prevent and treat infections of plants by bacteria, phytoplasmas, mycoplasmas, or fungi are disclosed in PCT Patent Application Publication WO2003/020024, which is incorporated by reference herein. Accordingly, the present disclosure further provides the use of any of the above compositions in the treatment or prevention of a plant infection.

Other plant infection that may be treated or prevented in accordance with the present disclosure may be caused by one or more of the following: Aspergillius fumigatus, Sclerotinia homeocarpa, Rhizoctonia solani, Colletotrichum graminicola, Phytophtora infestans, or Penicillium sp. As described herein, terpenes and/or the other therapeutic molecules, alone in suspension or solution may be somewhat unstable and may degrade rapidly in the soil environment, thus losing efficacy. Incorporation of a terpene or other therapeutic component in a hollow glucan particle or cell wall particle reduces the rate of release and degradation, thus increasing the duration of action of the molecule in the soil or on the plant. Accordingly, the terpene pro-payload and other components may be encapsulated as detailed above.

An advantage of a terpene based treatment of plants is that it can be applied shortly before harvest. Many conventional treatments require an extended period before re-entry to the treated area (generally 3 weeks). This means that an outbreak of a plant disease shortly before harvest cannot be treated with conventional treatments as it would then not be possible to harvest the crop at the desired time. The compositions of the present disclosure can suitably be applied at any time up until harvest, for example 21 days prior to harvest, 14 days prior to harvest, 7 days prior to harvest, or even 3 days or less before harvest. Prevention of plant infections can be achieved by treating plants which the compositions of the present disclosure regularly as a prophylactic measure.

Suitably, the composition of the present disclosure is applied by spraying. This is suitable for treating a plant disease which affects the surface of a plant. For spraying, a preparation comprising 2 g/l of the composition in water may be used. Concentrations of from 2 to 4 g/l are effective, and concentrations of greater than 4 g/l can be used as required. Obviously, it is important that the concentration of the composition used is sufficient to kill or inhibit the disease-causing agent, but not so high as to harm the plant being treated.

When spraying plants, a rate of 100 L/Ha or higher may generally be suitable to cover the plant. Typically, a rate of 100 to 500 L/Ha may be sufficient for crops of small plants which do not have extensive foliage; though higher rates may of course also be used as required. For larger plants with extensive foliage (e.g. perennial crop plants such as vines or other fruit plants) rates of 500 L/Ha or greater are generally suitable to cover the plants. A rate of 900 L/Ha or greater or 1200 L/Ha or greater is used to ensure good coverage. Where grape vines are being treated, a rate of 1200 L/Ha has proven suitably effective.

The composition of the present disclosure may alternatively be applied via irrigation. This is suitable for treating nematodes or other soil borne pathogens or parasites.

In certain embodiments, the present disclosure provides for compositions in the form of granules and methods of controlling pests using the same. Granules allow for the use of less selective herbicides, pesticides, and combinations thereof, and thus offer a means to control pests that are not otherwise easily controlled. Granules are a convenient application form for producers with small allotments such as paddy rice farmers, or for growers of turf where spays are complicated by the needs of near neighbors sensitive to drift or odor or for broad acre farmers who wish to apply fertilizers and herbicides together and who do not have convenient access to water.

The granules may be used in flooded paddies, recently irrigated turf, or in areas where it is inconvenient or impossible to remove irrigation water. The granules allow small holders the means to apply crop protection chemicals without expensive equipment, and without risk of exposing airways or eyes to aerosols or spray materials. Granules can be easily measured and distributed by hand. Using granules that are designed for uniform dispersal is advantageous because this compensates for uneven application.

### Pharmaceutical Compositions and Administration

In addition, the compositions and methods of the present disclosure are useful in the fields of industrial and consumer products and medicines, e.g., in food, human and animal drugs, and cosmetics, and the such. In some embodiments, the disclosure provides for compositions and methods for use in both human and veterinary medicine. In certain embodiments, the present disclosure relates to therapeutic treatment of mammals, birds, and fish. For example, the compositions and methods of the present disclosure are useful for therapeutic treatment of mammalian species including, but not limited to, human, bovine, ovine, porcine, equine, canine, and feline species.

Routes of administration of the delivery system include but are not limited to oral, buccal, sublingual, pulmonary, transdermal, transmucosal, as well as subcutaneous, intraperitoneal, intravenous, and intramuscular injection. Exemplary routes of administration are oral, buccal, sublingual, pulmonary, and transmucosal.

The particulate delivery system of the present disclosure is administered to a patient in a therapeutically effective amount. The particulate delivery system can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using a controlled release formulation. It is also noted that the dose of the compound can be varied over time. The particulate delivery system can be administered using an immediate release formulation, or using a controlled release formulation, or combinations thereof. The term “controlled release” includes sustained release, delayed release, and combinations thereof, as well as release mediated by chemical (e.g., pH) and/or biological (e.g., enzyme) hydrolysis.

A pharmaceutical composition of the disclosure can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a patient or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the animal or human treated, and further depending upon the route by which the composition is to be administered. By way of example, the composition can comprise between 0.1% and 100% (w/w) active ingredient. A unit dose of a pharmaceutical composition of the disclosure will generally comprise from about 100 milligrams to about 2 grams of the active ingredient, or from about 200 milligrams to about 1.0 gram of the active ingredient.

In addition, a particulate delivery system of the present disclosure can be administered alone, in combination with a particulate delivery system with a different payload, or with other pharmaceutically active compounds. The other pharmaceutically active compounds can be selected to treat the same condition as the particulate delivery system or a different condition.

If the patient is to receive or is receiving multiple pharmaceutically active compounds, the compounds can be administered simultaneously or sequentially in any order. For example, in the case of tablets, the active compounds may be found in one tablet or in separate tablets, which can be administered at once or sequentially in any order. In addition, it should be recognized that the compositions can be different forms. For example, one or more compounds may be delivered via a tablet, while another is administered via injection or orally as a syrup.

Another aspect of the disclosure relates to a kit comprising a pharmaceutical composition of the disclosure and instructional material. Instructional material includes a publication, a recording, a diagram, or any other medium of expression which is used to communicate the usefulness of the pharmaceutical composition of the disclosure for one of the purposes set forth herein in a human. The instructional material can also, for example, describe an appropriate dose of the pharmaceutical composition of the disclosure. The instructional material of the kit of the disclosure can, for example, be affixed to a container which contains a pharmaceutical composition of the disclosure or be shipped together with a container which contains the pharmaceutical composition. Alternatively, the instructional material can be shipped separately from the container with the intention that the instructional material and the pharmaceutical composition be used cooperatively by the recipient.

The disclosure also includes a kit comprising a pharmaceutical composition of the disclosure and a delivery device for delivering the composition to a human. By way of example, the delivery device can be a squeezable spray bottle, a metered-dose spray bottle, an aerosol spray device, an atomizer, a dry powder delivery device, a self-propelling solvent/powder-dispensing device, a syringe, a needle, a tampon, or a dosage-measuring container. The kit can further comprise an instructional material as described herein.

For example, a kit may comprise two separate pharmaceutical compositions comprising respectively a first composition comprising a particulate delivery system and a pharmaceutically acceptable carrier; and composition comprising second pharmaceutically active compound and a pharmaceutically acceptable carrier. The kit also comprises a container for the separate compositions, such as a divided bottle or a divided foil packet. Additional examples of containers include, without limitation, syringes, boxes, and bags. Typically, a kit comprises directions for the administration of the separate components. The kit form is advantageous when the separate components are administered in different dosage forms (e.g., oral and parenteral), are administered at different dosage intervals, or when titration of the individual components of the combination is desired by the prescribing physician.

An example of a kit is a blister pack. Blister packs are well known in the packaging industry and are being widely used for the packaging of pharmaceutical unit dosage forms, e.g., tablets and capsules. Blister packs generally consist of a sheet of relatively stiff material covered with a foil of, e.g., a transparent plastic material. During the packaging process, recesses are formed in the plastic foil. The recesses have the size and shape of the tablets or capsules to be packed. Next, the tablets or capsules are placed in the recesses and a sheet of relatively stiff material is sealed against the plastic foil at the face of the foil which is opposite from the direction in which the recesses were formed. As a result, the tablets or capsules are sealed in the recesses between the plastic foil and the sheet. The strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen that the tablets or capsules so specified should be ingested. Another example of such a memory aid is a calendar printed on the card, e.g., as follows “First Week, Monday, Tuesday,. etc. Second Week, Monday, Tuesday,” etc. Other variations of memory aids will be readily apparent. A “daily dose” can be a single tablet or capsule or several pills or capsules to be taken on a given day. Also, a daily dose of a particulate delivery system composition can consist of one tablet or capsule, while a daily dose of the second compound can consist of several tablets or capsules and vice versa. The memory aid should reflect this and assist in correct administration.

In another embodiment of the present disclosure, a dispenser designed to dispense the daily doses one at a time in the order of their intended use is provided. The dispenser may be equipped with a memory aid, so as to further facilitate compliance with the dosage regimen. An example of such a memory aid is a mechanical counter, which indicates the number of daily doses that have been dispensed. Another example of such a memory aid is a battery-powered micro-chip memory coupled with a liquid crystal readout, or audible reminder signal which, for example, reads out the date that the last daily dose has been taken and/or reminds one when the next dose is to be taken.

A particulate delivery system composition, optionally comprising other pharmaceutically active compounds, can be administered to a patient either orally, rectally, parenterally, (for example, intravenously, intramuscularly, or subcutaneously) intracisternally, intravaginally, intraperitoneally, intravesically, locally (for example, powders, ointments or drops), or as a buccal or nasal spray.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound. Parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise the active ingredient combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols, e.g., propylene glycol, polyethylene glycol, and glycerol, and suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, the active ingredient is provided in dry (e.g., powder or granular) form for reconstitution with a suitable vehicle (e.g., sterile pyrogen-free water) prior to parenteral administration of the reconstituted composition. The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using a non-toxic parenterally-acceptable diluent or solvent, such as water or 1,3-butanediol, for example. Other acceptable diluents and solvents include Ringer's solution, isotonic sodium chloride solution, and fixed oils such as synthetic mono-or di-glycerides. Other parentally-administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, and sorbic acid. It may also be desirable to include isotonic agents, for example, sugars, and sodium chloride. Prolonged absorption of injectable pharmaceutical compositions can be brought about by the use of agents capable of delaying absorption, for example, aluminum monostearate and/or gelatin.

Dosage forms can include solid or injectable implants or depots. In certain embodiments, the implant comprises an aliquot of the particulate delivery system and a biodegradable polymer. In certain embodiments, a suitable biodegradable polymer can be selected from the group consisting of a polyaspartate, polyglutamate, poly(L-lactide), a poly(D, L-lactide), a poly(lactide-co-glycolide), a poly(ε-caprolactone), a polyanhydride, a poly(beta-hydroxy butyrate), a poly(ortho ester), and a polyphosphazene.

Solid dosage forms for oral administration include capsules, tablets, powders, and granules. In such solid dosage forms, the particulate delivery system is optionally admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the particulate delivery system can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets can be made by molding, in a suitable device, a mixture of the active ingredient, a pharmaceutically acceptable carrier, and at least sufficient liquid to moisten the mixture. Pharmaceutically acceptable excipients used in the manufacture of tablets include inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or they can be coated using known methods to achieve delayed disintegration in the gastrointestinal tract of a human, thereby providing sustained release and absorption of the particulate delivery system, e.g. in the region of the Peyer's patches in the small intestine. By way of example, a material such as glyceryl monostearate or glyceryl distearate can be used to coat tablets. Further by way of example, tablets can be coated using methods described in U.S. Pat. Nos. 4,256,108; 4,160,452; and 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents, and can also be of such composition that they release the particulate delivery system in a delayed manner. Examples of embedding compositions that can be used are polymeric substances and waxes. The active compounds can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols. Hard capsules comprising the particulate delivery system can be made using a physiologically degradable composition, such as gelatin. Such hard capsules comprise the particulate delivery system, and can further comprise additional ingredients including, for example, an inert solid diluent such as calcium carbonate, calcium phosphate, or kaolin. Soft gelatin capsules comprising the particulate delivery system can be made using a physiologically degradable composition, such as gelatin. Such soft capsules comprise the particulate delivery system, which can be mixed with water or an oil medium such as peanut oil, liquid paraffin, or olive oil.

Oral compositions can be made, using known technology, which specifically release orally-administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (see PCT application PCT/GB89/00581) can be used in such formulations.

Pulsed release technology such as that described in U.S. Pat. No. 4,777,049 can also be used to administer the particulate delivery system to a specific location within the gastrointestinal tract. Such systems permit delivery at a predetermined time and can be used to deliver the particulate delivery system, optionally together with other additives that my alter the local microenvironment to promote stability and uptake, directly without relying on external conditions other than the presence of water to provide in vivo release.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, isotonic saline, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, e.g., almond oil, arachis oil, coconut oil, cottonseed oil, groundnut oil, corn germ oil, olive oil, castor oil, sesame seed oil, MIGLYOL™, glycerol, fractionated vegetable oils, mineral oils such as liquid paraffin, tetrahydrofurfuryl alcohol, polyethylene glycols, fatty acid esters of sorbitan, or mixtures of these substances. Besides such inert diluents, the composition can also include adjuvants, such as wetting agents, emulsifying and suspending agents, demulcents, preservatives, buffers, salts, sweetening, flavoring, coloring and perfuming agents. Suspensions, in addition to the active compound, may contain suspending agents, as for example, ethoxylated isostearyl alcohols, polyoxyethylene sorbitol or sorbitan esters, microcrystalline cellulose, hydrogenated edible fats, sodium alginate, polyvinylpyrrolidone, gum tragacanth, gum acacia, agar-agar, and cellulose derivatives such as sodium carboxymethylcellulose, methylcellulose, hydroxypropylmethylcellulose, aluminum metahydroxide, bentonite, or mixtures of these substances. Liquid formulations of a pharmaceutical composition of the disclosure that are suitable for oral administration can be prepared, packaged, and sold either in liquid form or in the form of a dry product intended for reconstitution with water or another suitable vehicle prior to use.

Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

For topical administration liquids, suspension, lotions, creams, gels, ointments, drops, suppositories, sprays and powders may be used. Conventional pharmaceutical carriers, aqueous, powder or oily bases, and thickeners can be used as necessary or desirable.

In other embodiments, the pharmaceutical composition can be prepared as a nutraceutical, i.e., in the form of, or added to, a food (e.g., a processed item intended for direct consumption) or a foodstuff (e.g., an edible ingredient intended for incorporation into a food prior to ingestion). Examples of suitable foods include candies such as lollipops, baked goods such as crackers, breads, cookies, and snack cakes, whole, pureed, or mashed fruits and vegetables, beverages, and processed meat products. Examples of suitable foodstuffs include milled grains and sugars, spices and other seasonings, and syrups. The particulate delivery systems described herein are not exposed to high cooking temperatures for extended periods of time, in order to minimize degradation of the compounds.

Compositions for rectal or vaginal administration can be prepared by mixing a particulate delivery system with suitable non-irritating excipients or carriers such as cocoa butter, polyethylene glycol or a suppository wax, which are solid at ordinary room temperature, but liquid at body temperature, and therefore, melt in the rectum or vaginal cavity and release the particulate delivery system. Such a composition can be in the form of, for example, a suppository, a retention enema preparation, and a solution for rectal or colonic irrigation. Suppository formulations can further comprise various additional ingredients including antioxidants and preservatives. Retention enema preparations or solutions for rectal or colonic irrigation can be made by combining the active ingredient with a pharmaceutically acceptable liquid carrier. As is known in the art, enema preparations can be administered using, and can be packaged within, a delivery device adapted to the rectal anatomy of a human. Enema preparations can further comprise various additional ingredients including antioxidants and preservatives.

A pharmaceutical composition of the disclosure can be prepared, packaged, or sold in a formulation suitable for pulmonary administration via the buccal cavity. Such compositions are conveniently in the form of dry powders for administration using a device comprising a dry powder reservoir to which a stream of propellant can be directed to disperse the powder or using a self-propelling solvent/powder-dispensing container such as a device comprising the particulate delivery system suspended in a low-boiling propellant in a sealed container. Dry powder compositions may include a solid fine powder diluent such as sugar and are conveniently provided in a unit dose form. Low boiling propellants generally include liquid propellants having a boiling point below 65 degrees F. at atmospheric pressure. Generally the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent, e.g., having a particle size of the same order as particles comprising the particulate delivery system.

Pharmaceutical compositions of the disclosure formulated for pulmonary delivery can also provide the active ingredient in the form of droplets of a suspension. Such formulations can be prepared, packaged, or sold as aqueous or dilute alcoholic suspensions, optionally sterile, comprising the particulate delivery system, and can conveniently be administered using any nebulization or atomization device. Such formulations can further comprise one or more additional ingredients including a flavoring agent such as saccharin sodium, a volatile oil, a buffering agent, a surface-active agent, or a preservative such as methylhydroxybenzoate.

The formulations described herein as being useful for pulmonary delivery are also useful for intranasal delivery of a pharmaceutical composition of the disclosure. Another formulation suitable for intranasal administration is a coarse powder comprising the particulate delivery system. Such a formulation is administered in the manner in which snuff is taken, i.e., by rapid inhalation through the nasal passage from a container of the powder held close to the nares.

A pharmaceutical composition of the disclosure can be prepared, packaged, or sold in a formulation suitable for buccal administration. Such formulations can, for example, be in the form of tablets or lozenges made using conventional methods, and can, for example, comprise 0.1 to 20% (w/w) particulate delivery system, the balance comprising an orally dissolvable or degradable composition and, optionally, one or more of the additional ingredients described herein. Alternately, formulations suitable for buccal administration can comprise a powder or an aerosolized or atomized solution or suspension comprising the particulate delivery system.

Reference will now be made to specific examples illustrating the disclosure. It is to be understood that the examples are provided to illustrate exemplary embodiments and that no limitation to the scope of the disclosure is intended thereby.

### EXAMPLES

### Example 1: Yeast Cell Wall Particle Production

Exemplary extracted YCWPs are readily available, biodegradable, substantially spherical particles about 2-4 μm in diameter. Preparation of extracted yeast cell wall particles is known in the art, and is described, for example in U.S. Pat. Nos. 4,992,540, 5,082,936, 5,028,703, 5,032,401, 5,322,841, 5,401,727, 5,504,079, 5,968,811, 6,444,448 B1, 6,476,003B1, published U.S. applications 2003/0216346 A1, 2004/0014715 A1, and published PCT application WO 02/12348 A2, the teachings of which are incorporated herein by reference.

A form of extracted yeast cell wall particles, referred to as “whole glucan particles” or “WPGs” (See U.S. Pat. Nos. 5,032,401 and 5,607,677), may be modified to facilitate improved retention and/or delivery of payload molecules. Such improvements build on the art-recognized WGPs but feature trapping molecules and nanoparticles as well as pluralities of said trapping molecules and nanoparticles, formulated in specific forms to achieve the desired improved delivery properties. As used herein, a WGP is typically a whole glucan particle of >90% beta glucan purity.

### Preparation of Glucan Particles (GPS)

Glucan particles (GPs), also referred to herein as yeast glucan particles (“YGPs”), are a purified hollow yeast cell ‘ghost’ containing a rich β-glucan sphere, generally 2-4 microns in diameter. GPs have been used for macrophage-targeted delivery of soluble payloads (DNA, siRNA, protein, and small molecules) encapsulated inside the hollow GPs via core polyplex and layer-by-layer (LbL) synthetic strategies.

In general, glucan particles can be prepared from yeast cells by the extraction and purification of the alkali-insoluble glucan fraction from the yeast cell walls. The yeast cells can be treated with an aqueous hydroxide solution without disrupting the yeast cell walls, which digests the protein and intracellular portion of the cell, leaving the glucan wall component devoid of significant protein contamination, and having substantially the unaltered cell wall structure of β(1-6) and β(1-3) linked glucans. The 1,3-β-glucan outer shell provides for receptor-mediated uptake by phagocytic cells, e.g., macrophages, expressing β-glucan receptors.

Certain glucan particles, can be made as follows: yeast particles (S. cerevisae), Biorigin MOS55 are suspended in 1 liter of 1M NaOH and heated to 85° C. The cell suspension can be stirred vigorously for 1 hour at this temperature. The insoluble material containing the cell walls can be recovered by centrifuging. This material can be then suspended in 1M NaOH, heated, and stirred vigorously for 1 hour. The suspension can be then allowed to cool to room temperature and the extraction can be continued for a further 16 hours. The insoluble residue can be recovered by centrifugation. This material can be finally extracted in water brought to pH 4.5 with HCl. The insoluble residue can be recovered by centrifugation and washed three times with water, isopropanol, and acetone. The resulting slurry can be placed in glass trays and dried under reduced pressure to produce a fine white powder.

A more detailed description of processes for preparing WPGs can be found in U.S. Pats. Nos. 4,810,646, 4,992,540, 5,028,703, 5,607,677, and 5,741,495 (incorporated herein by reference). For example, U.S. Pat. No. 5,028,703 discloses that yeast WGP particles can be produced from yeast strain R4 cells in fermentation culture. The cells can be harvested by batch centrifugation at 8000 rpm for 20 minutes in a Sorval RC2-B centrifuge. The cells can be then washed twice in distilled water in order to prepare them for the extraction of the whole glucan. The first step involved resuspending the cell mass in 1 liter 4% w/v NaOH and heating to 100° C. The cell suspension can be stirred vigorously for 1 hour at this temperature. The insoluble material containing the cell walls can be recovered by centrifuging at 2000 rpm for 15 minutes. This material can be then suspended in 2 liters, 3% w/v NaOH and heated to 75° C. The suspension can be stirred vigorously for 3 hours at this temperature. The suspension can be then allowed to cool to room temperature and the extraction can be continued for a further 16 hours. The insoluble residue can be recovered by centrifugation at 2000 rpm for 15 minutes. This material can be finally extracted in 2 liters, 3% w/v NaOH brought to pH 4.5 with HCl, at 75° C. for 1 hour. The insoluble residue can be recovered by centrifugation and washed three times with 200 milliliters water, once with 200 milliliters dehydrated ethanol, and twice with 200 milliliters dehydrated ethyl ether. The resulting slurry can be placed on petri plates and dried.

Varying degrees of purity of glucan particles can be achieved by modifying the extraction/purification process. As used herein, the terms YCWP, YGP, and GP describe a 2-4 micron hollow microsphere (or yeast cell wall ghost) purified from Baker's yeast using a series of alkaline, acid, and organic extraction steps as detailed supra. In general, these GPs are on the order of 80-85% pure on a w/w basis of beta glucan and, following the introduction of payload, trapping, or other components, become of a slightly lesser “purity.” In exemplary embodiments, GPs are <90% beta glucan purity.

### Preparation of YCP Particles

Yeast cells (Rhodotorula sp.) derived from cultures obtained from the American Type Culture Collection (ATCC, Manassas, Va.) can be aerobically grown to stationary phase in YPD at 30° C. Rhodotorula sp. cultures available from ATCC include Nos. 886, 917, 9336, 18101, 20254, 20837 and 28983. Cells can be harvested by batch centrifugation at 2000 rpm for 10 minutes. The cells can be then washed once in distilled water and then re-suspended in water brought to pH 4.5 with HCl, at 75° C. for 1 hour. The insoluble material containing the cell walls can be recovered by centrifuging. This material can be then suspended in 1 liter, 1M NaOH and heated to 90° C. for 1 hour. The suspension can be then allowed to cool to room temperature and the extraction can be continued for a further 16 hours. The insoluble residue can be recovered by centrifugation and washed twice with water, isopropanol, and acetone. The resulting slurry can be placed in glass trays and dried at room temperature to produce 2.7 g of a fine light brown powder.

In alternative embodiments, YGPs, e.g., activated YGPs, can be grafted with chitosan on the surface, for example, to increase total surface chitosan. Chitosan can further be acetylated to form chitin (YGCP), in certain embodiments. Such particles can be seen to have equivalent properties when seen, in vivo, by the immune system of a subject or patient.

### Preparation of YGMP Particles

S. cerevisiae (100 g Fleishmans Baker's yeast) can be suspended in 1 liter 1M NaOH and heated to 55° C. The cell suspension can be mixed for 1 hour at this temperature. The insoluble material containing the cell walls can be recovered by centrifuging at 2000 rpm for 10 minutes. This material can be then suspended in 1 liter of water and brought to pH 4-5 with HCl, and incubated at 55° C. for 1 hour. The insoluble residue can be recovered by centrifugation and washed once with 1000 milliliters water, four times with 200 milliliters dehydrated isopropanol and twice with 200 milliliters acetone. The resulting slurry can be placed in a glass tray and dried at room temperature to produce 12.4 g of a fine, slightly off-white, powder.
S. cerevisiae (75 g SAF-Mannan) can be suspended in 1 liter water and adjusted to pH 12-12.5 with 1M NaOH and heated to 55° C. The cell suspension can be mixed for 1 hour at this temperature. The insoluble material containing the cell walls can be recovered by centrifuging at 2000 rpm for 10 minutes. This material can be then suspended in 1 liter of water and brought to pH 4-5 with HCl, and incubated at 55° C. for 1 hour. The insoluble residue can be recovered by centrifugation and washed once with water, dehydrated isopropanol, and acetone. The resulting slurry can be placed in a glass tray and dried at room temperature to produce 15.6 g of a fine slightly off-white powder.

### Example 2: Synthesis of Controlled-Release Pro-Payload Molecules

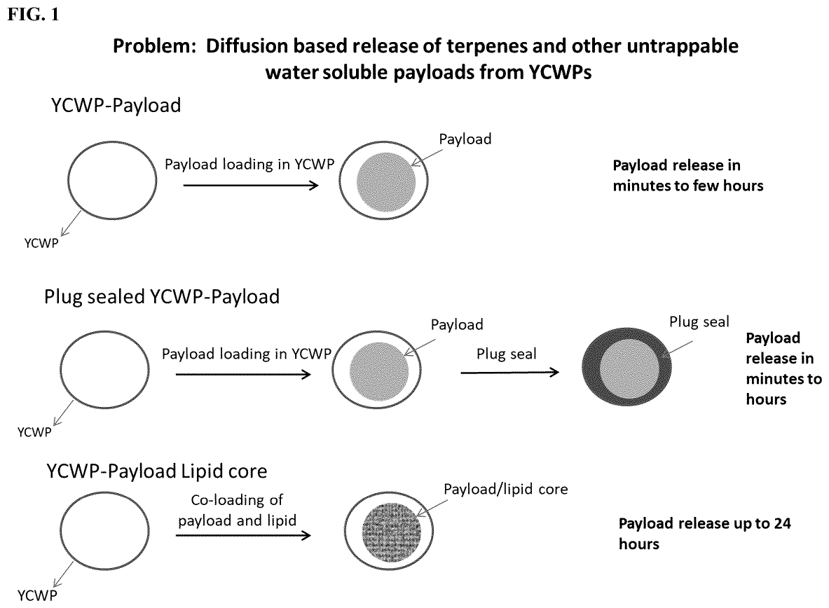

Water-soluble payloads that cannot be trapped by any of the existing methods, can be loaded inside yeast cell wall particles, but their release is characterized by rapid diffusion out of the YCWPs. Several approaches have been attempted to improve payload retention by using polymer-based gels to plug seal the yeast cell wall particles, and by co-loading of payloads with a hydrophobic lipid material to form a payload/lipid core that slows diffusion of the payload from the YCWP. These approaches are shown schematically in FIG. 1. However, since none of these methods retain highly-soluble payload molecules for longer than twenty-four hours, there is a need in the art for the development of compositions and methods for delivering water-soluble payloads to cells in a controlled manner.

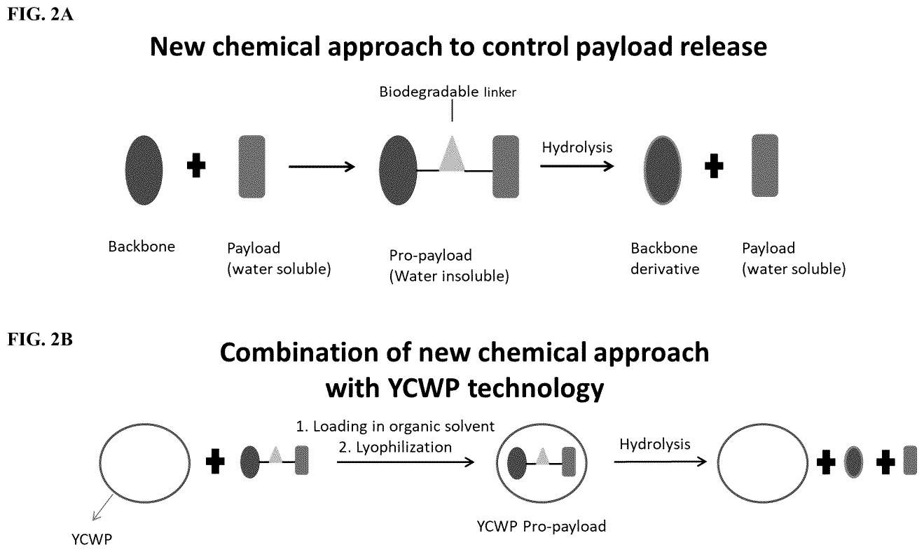

In this disclosure, untrappable water-soluble payloads are chemically modified to yield water-insoluble payload derivatives, i.e., pro-payloads, shown in FIG. 2A. These water-insoluble pro-payloads contain a bond that is susceptible to chemical (e.g., pH) and/or biological (e.g., enzymatic) hydrolysis that regenerates the water-soluble payload, shown in FIG. 2B. The pro-payloads may consist of a wide range of structural materials, i.e., scaffolding molecules and linkers, that are well known in the art, including esters and polyesters, acetals, carbamates, disulfides, cycloalkanes, polyphosphates, aminocarboxylic acids, hydrazines, hydrazones, amides, and anhydrides.

To create encapsulated pro-payloads, water-insoluble pro-payloads are dissolved in a suitable solvent compatible with the glucan particles (e.g., dimethylsulfoxide, methanol, etc.). After loading the pro-payloads into glucan shells, the glucan particle pro-payload is processed to remove the solvent. Encapsulation efficiencies in excess of 90% have been observed using the instant methods.

Because pro-payloads are water-insoluble and have slow hydrolysis at neutral pH, the YCWP pro-payload technology offers improved payload stability. Moreover, payload release can be precisely controlled through chemical or enzymatic hydrolysis of the biodegradable linker.

### Example 3: Creation and Characterization of Pro-Payloads Comprising Ester as a Biodegradable Linker for Payloads with Hydroxyl Groups

### A. Dicarvacrol Edta

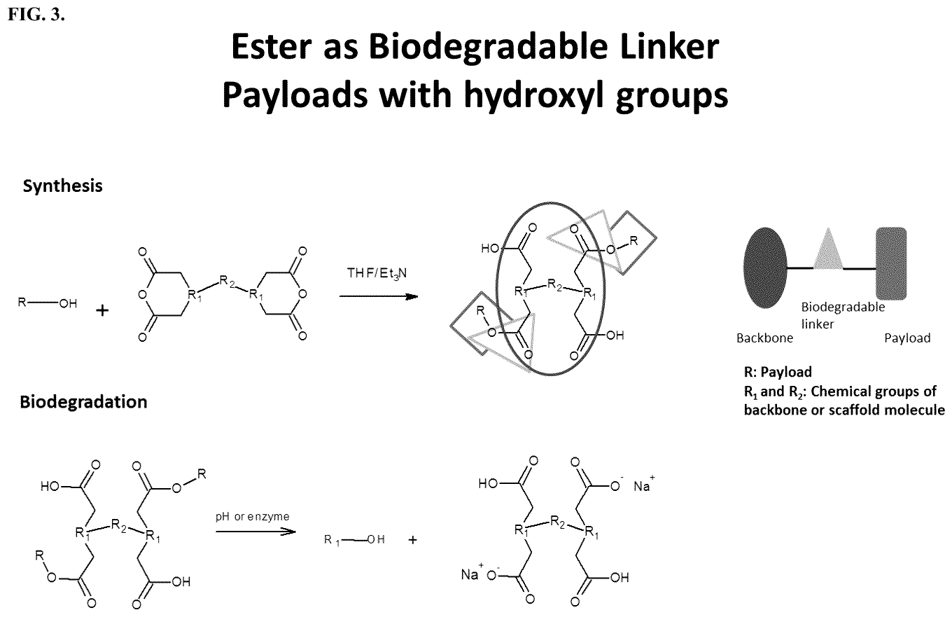

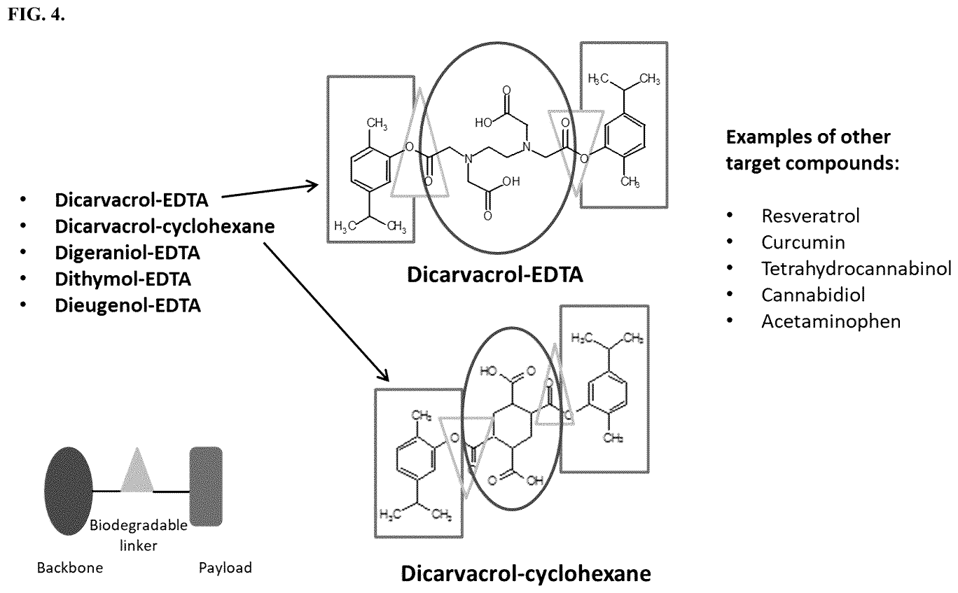

FIG. 3 shows an example of a pro-payload molecule with an ester biodegradable linker generated from a payload with a hydroxyl group. Carvacrol (C₆H₃CH₃(OH)(C₃H₇)) is a water-soluble monoterpenoid phenol with antimicrobial properties. FIG. 4 shows examples of dicarvacrol-EDTA (ethylenediaminetetraacetic acid) and dicarvacrol-cyclohexane-1,4-dicarboxylic acid pro-payload molecules synthesized to contain cleavable linkers. Pro-payload molecules comprising, e.g., resveratrol, curcumin, tetrahydrocannabinol, cannabidiol, and acetaminophen, may also be generated according to the methods described.

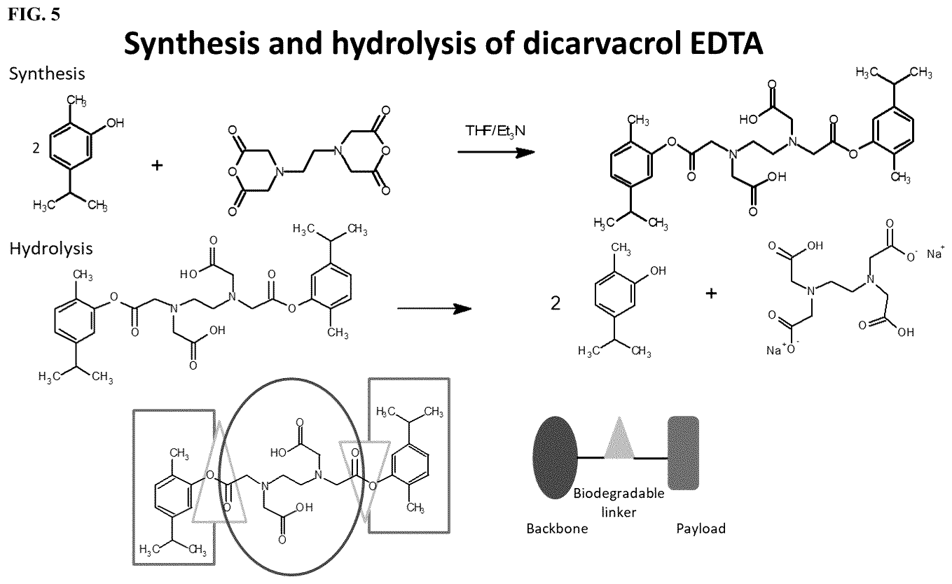

The water-insoluble pro-payload dicarvacrol-EDTA was synthesized by reacting carvacrol with the scaffolding payload molecule ethylenediaminetetraacetic dianhydride. As shown in FIG. 5, the addition of two carvacrol payload molecules to one molecule of EDTA dianhydride, in tetrahydrofuran, was catalyzed by triethylamine. The resulting ester linkages (shown schematically as yellow triangles) may be hydrolyzed by enzymatic cleavage, e.g., with an esterase, thereby releasing the two carvacrol payload molecules (shown schematically as green rectangles) from the EDTA backbone (shown schematically as a red oval).

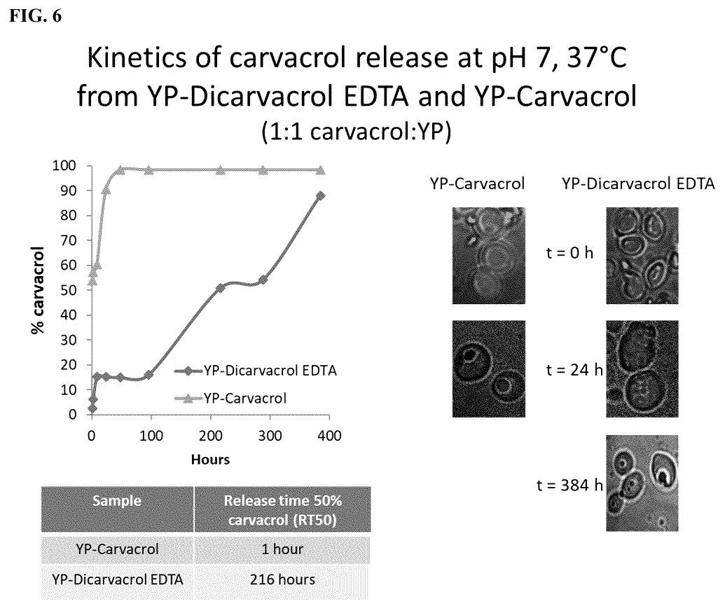

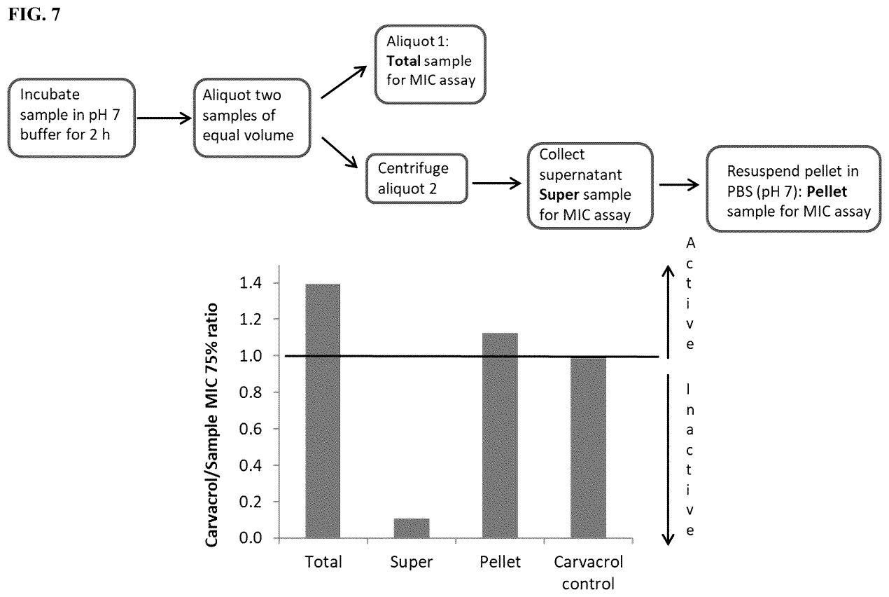

The pro-payload dicarvacrol-EDTA was dissolved in a suitable organic solvent and combined with extracted yeast cell wall particles in a 1:1 weight ratio to create encapsulated dicarvacrol-EDTA. As shown in FIG. 6, encapsulated pro-payload dicarvacrol-EDTA (YP-Dicarvacrol-EDTA) demonstrated vastly improved diffusion kinetics compared to carvacrol which had been encapsulated in yeast cell particles using conventional means (YP-Carvacrol). In an in vitro release assay, YP-dicarvacrol-EDTA had a 50% release time (RT50) of 216 hours compared to one (1) hour for YP-carvacrol. Nearly all of the carvacrol was released from the YP-carvacrol sample within 24 hours, whereas more than 10% of the carvacrol was present in the YP-dicarvacrol-EDTA sample 384 hours after assay commencement. These data demonstrate how the generation of pro-payloads from soluble payload molecules effectively prevents the release of payloads from encapsulating yeast cell wall particles. Compared to its carvacrol control, YP-dicarvacrol-EDTA remained functionally stable at pH 7.0, and retained its antimicrobial activity against E. coli in an in vitro bacterial inhibition assay, as shown in the pellet fraction vs. the control and supernatant fractions of FIG. 7.

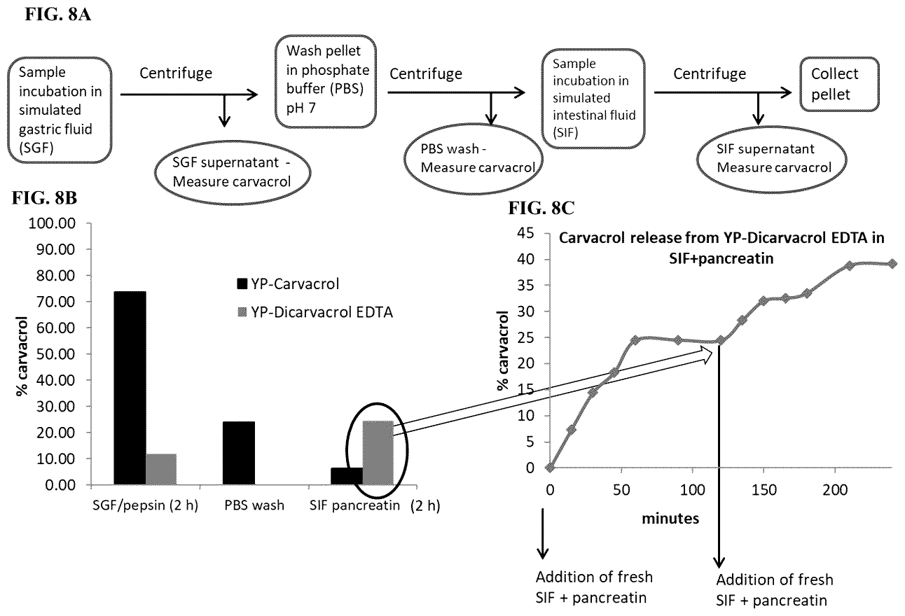

FIG. 8A summarizes an in vitro payload release assay that was performed to determine whether orally-delivered carvacrol payloads could be selectively delivered to the intestines of an animal. In a first step, YP-dicarvacrol-EDTA and YP-carvacrol were incubated in a simulated mammalian gastric fluid (SGF) solution containing pepsin. The incubated samples were then centrifuged and carvacrol was measured in the supernatant. As shown in FIG. 8B, approximately 65% more free carvacrol was observed in the YP-carvacrol SGF-digested sample than the YP-dicarvacrol-EDTA SGF-digested sample, indicating that the chemical linkage in YP-dicarvacrol-EDTA is resistant to digestion by gastric fluids. Following digestion with SGF, the samples were further tested for stability in simulated intestinal fluid (SIF) containing pancreatin. The SGF-digested samples were centrifuged, washed in phosphate buffered saline (PBS), and incubated in SIF. As shown in FIG. 8C, the addition of SIF resulted in the release of more carvacrol from the YP-dicarvacrol-EDTA sample than from YP-carvacrol, indicating that intestinal fluids are capable of hydrolyzing the chemical linkage in YP-dicarvacrol-EDTA. Thus, pro-payloads can be designed to effectively and selectively deliver payload molecules to the digestive systems, e.g., the intestines of animals.

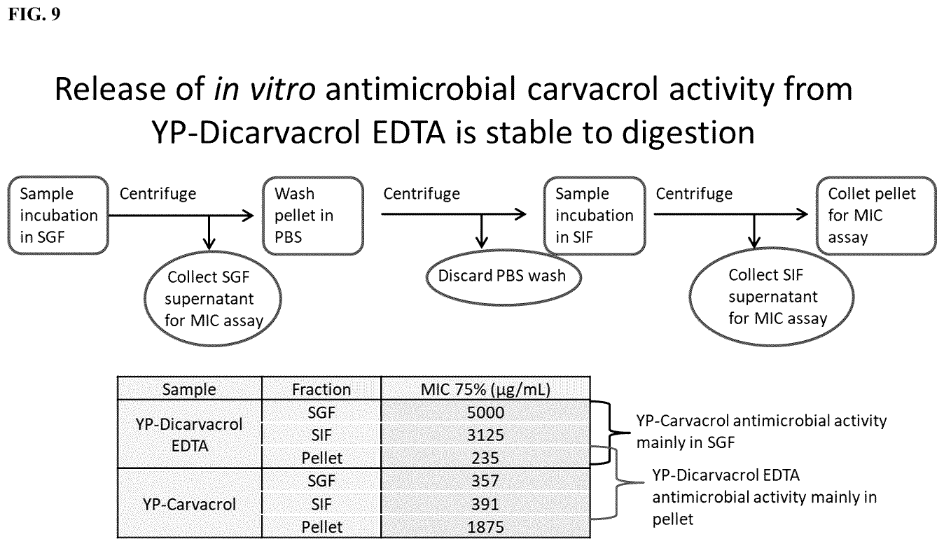

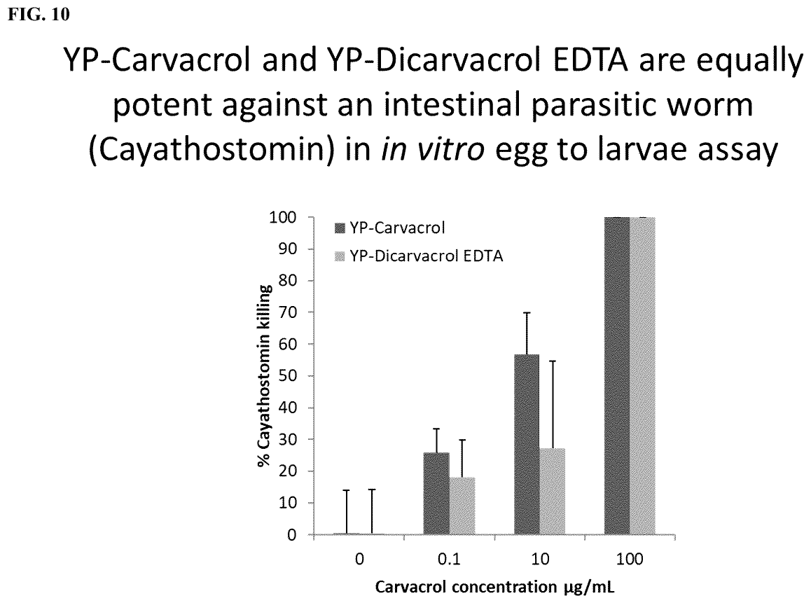

To determine whether encapsulated and released carvacrol retained antimicrobial activity, pelleted and soluble fractions were collected from YP-dicarvacrol-EDTA and YP-carvacrol samples that had been incubated in SGF and SIF. The fractions were then tested for their ability to inhibit E. coli in an in vitro bacterial inhibition assay. As shown in FIG. 9, antimicrobial activity was localized in the SGF supernatant fraction of the digested YP-carvacrol sample, whereas antimicrobial activity was localized in the pelleted fraction of the YP-dicarvacrol-EDTA sample. As shown in FIG. 10, YP-dicarvacrol-EDTA and YP-carvacrol were equally effective against an intestinal parasitic worm (i.e., Cayathostomin) in an in vitro egg to larvae assay. A dosage of 10 μg/ml carvacrol effectively killed approximately 50% of Cayathostomin eggs/larva, whereas the 100 μg/ml dosage was 100% effective. These data further demonstrate the efficacy of pro-payloads for treating microbial and parasitic infections in animals.

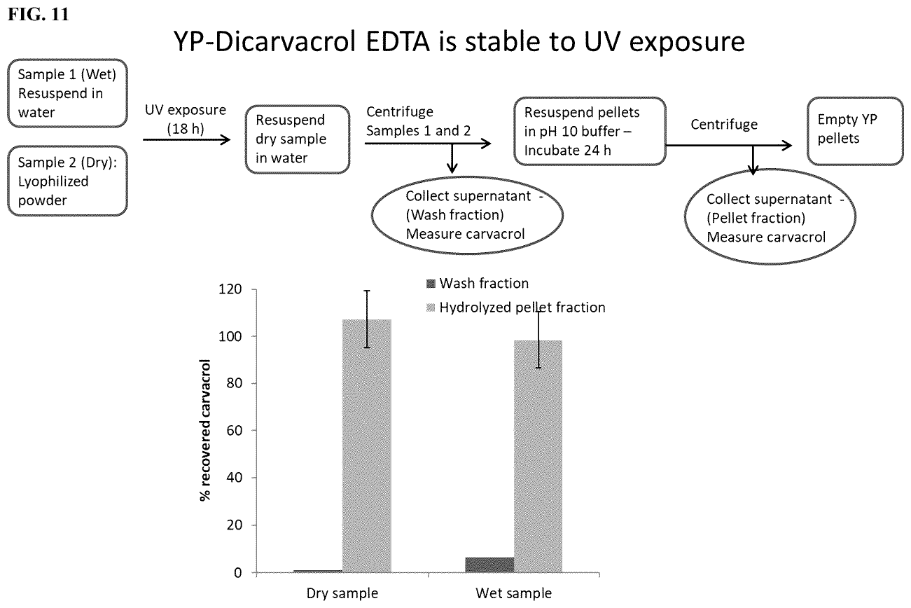

In addition to demonstrating improved stability of pro-payloads in simulated gastric fluid, the YP-dicarvacrol-EDTA was stable in the presence of ultraviolet (UV) light radiation. Importantly, as shown in FIG. 11, this improved stability was observed in both lyophilized and aqueous samples of YP-dicarvacrol-EDTA, indicating that pro-payloads can be formulated and stored as both solid and aqueous compositions (pH 7) without concern for exposure to light or UV radiation.

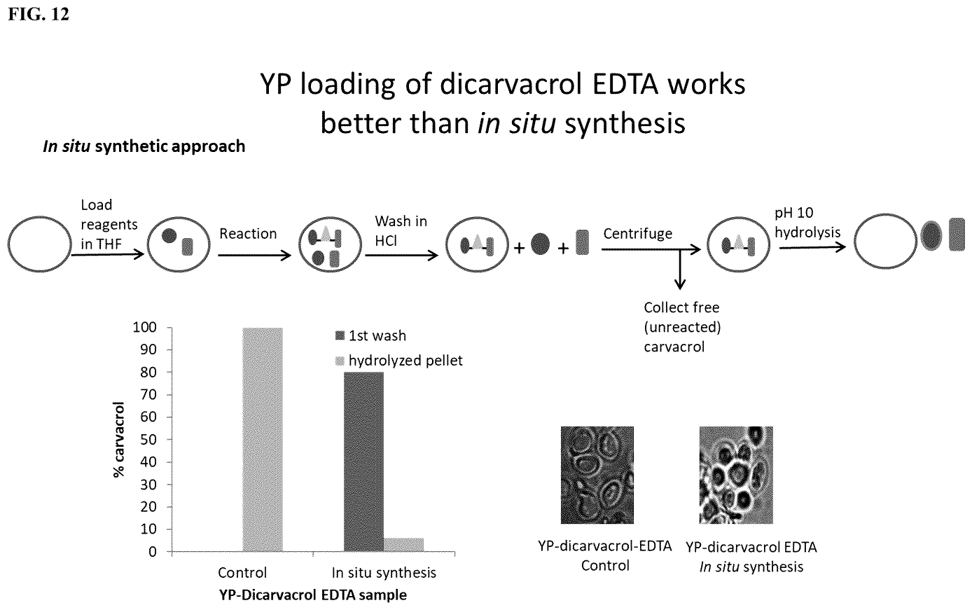

The encapsulated pro-payloads described above were created by first generating pro-payloads by reacting two payload molecules with a payload scaffolding molecule, followed by suspending the pro-payloads in a suitable organic solvent, and then combining them with extracted yeast cell wall particles in a 1:1 weight ratio to create encapsulated pro-payloads. To determine whether encapsulated pro-payloads could be generated in situ using fewer steps, carvacrol, EDTA, and extracted yeast cell wall particles were incubated together in tetrahydrofuran following the process shown schematically in FIG. 12. As demonstrated, considerable free carvacrol was observed in the wash fraction following the in situ reaction, indicating that the sequential loading of pre-formed pro-payloads into yeast cell wall particles is more effective than in situ synthesis.

### B. Dicarvacrol-Cyclohexane (DCC6)

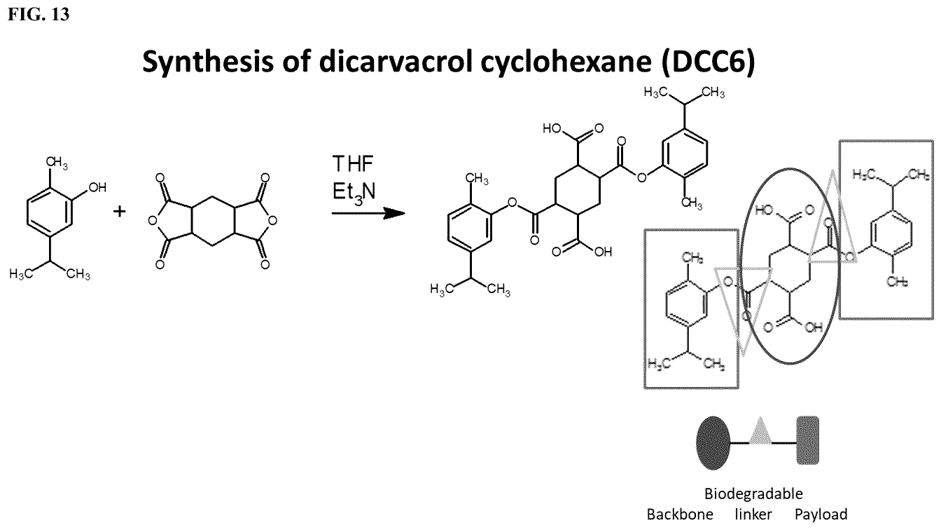

The water-insoluble pro-payload dicarvacrol-cyclohexane-1,4-dicarboxylic acid (YP-DCC6) was synthesized by reacting the payload carvacrol with the scaffolding payload molecules 1,2,4,5-cyclohexane tetracarboxylic dianhydride. As shown in FIG. 13, the addition of two carvacrol payload molecules to one molecule of 1,2,4,5-cyclohexane tetracarboxylic dianhydride, in tetrahydrofuran, was catalyzed by triethylamine. The resulting ester linkages (yellow triangles) may be hydrolyzed by enzymatic cleavage, e.g., with an esterase, thereby releasing the two carvacrol payload molecules (green rectangles) from their pro-payload form.

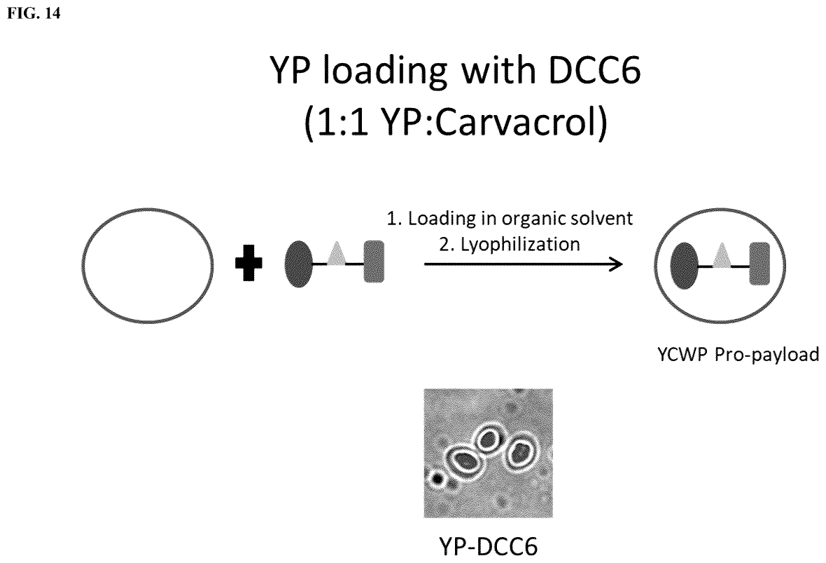

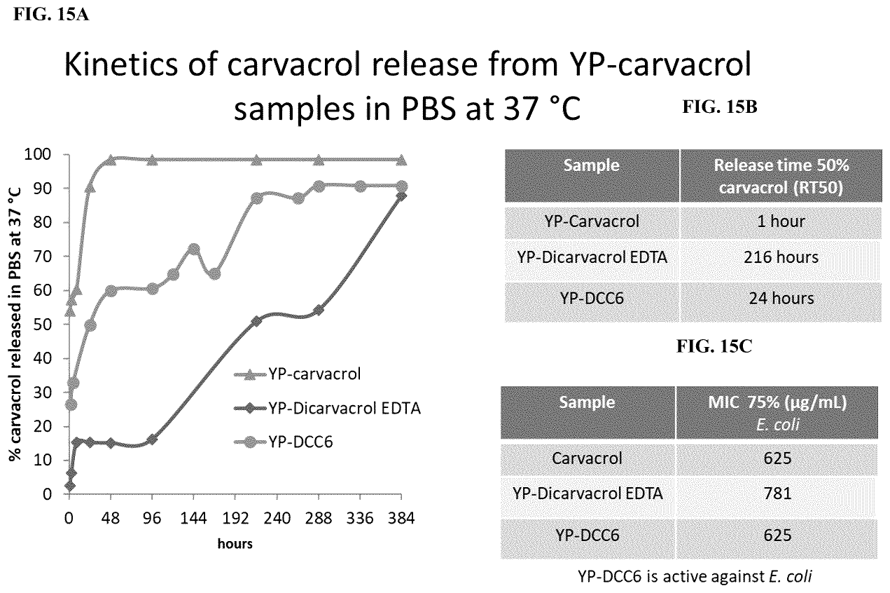

As shown in FIG. 14, the pro-payload dicarvacrol-cyclohexane-1,4-dicarboxylic acid (DCC6) was dissolved in a suitable organic solvent and combined with extracted yeast cell wall particles in a 1:1 weight ratio to create encapsulated dicarvacrol-cyclohexane-1,4-dicarboxylic acid (DCC6). As shown in FIGS. 15A and 15B, encapsulated YP-DCC6 and YP-dicarvacrol-EDTA demonstrated improved diffusion kinetics compared to carvacrol, which had been encapsulated in yeast cell particles using conventional means (YP-Carvacrol). In an in vitro release assay, YP-DCC6 had a 50% release time (RT50) of 24 hours and YP-dicarvacrol-EDTA had an RT50 time of 216 hours, compared to one (1) hour for YP-carvacrol. All samples retained their antimicrobial abilities in an in vitro bacterial inhibition assay, shown in FIG. 15C.

### C. Di-Terpene EDTA Compounds

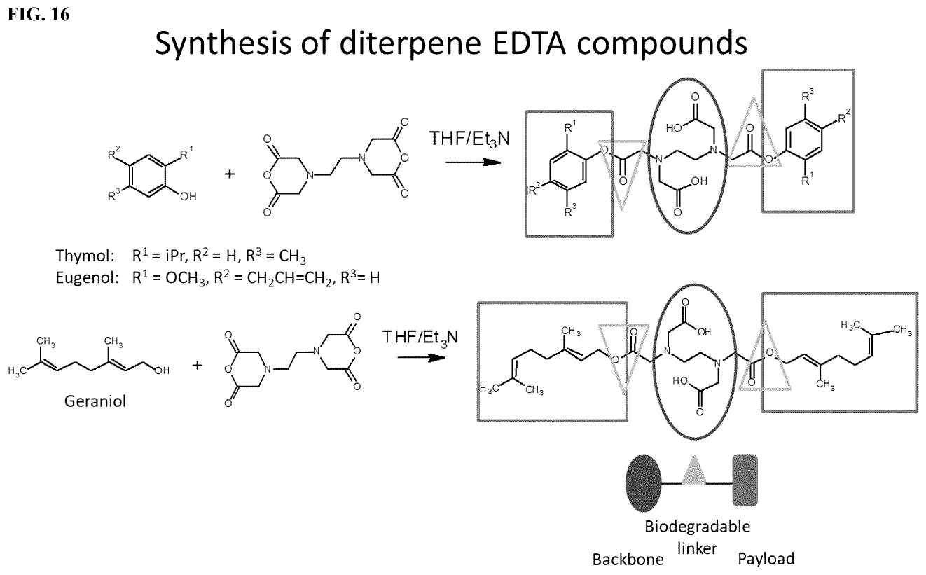

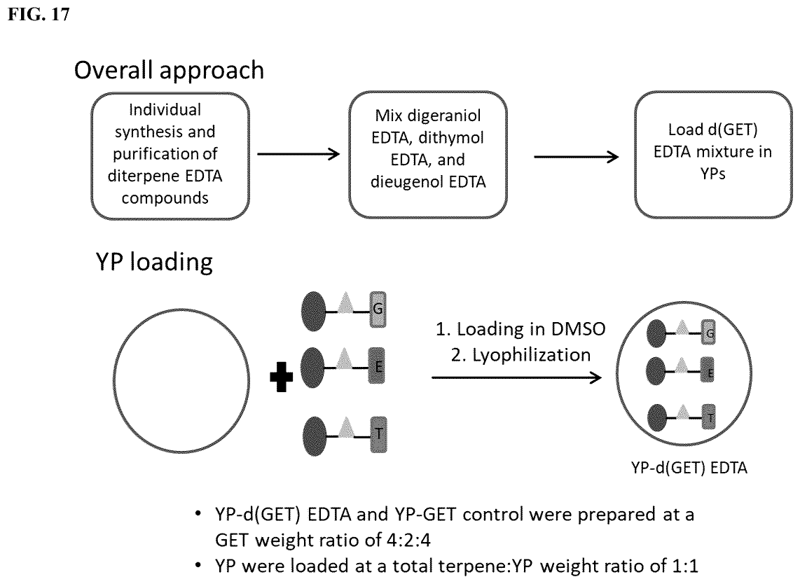

Geraniol, eugenol, and thymol are partially water soluble, naturally occurring terpenes or terpene-like molecules that possess a variety of antimicrobial, antifungal, and medicinal properties. Water-insoluble pro-payload di-terpene-EDTA compounds were synthesized by reacting the payload molecules geraniol, eugenol, and thymol with EDTA dianhydride. As shown in FIG. 16, the addition of two of each respective payload molecule to one molecule of EDTA, in THF, was catalyzed by triethylamine. The resulting pro-payloads were mixed in a ratio of 4:2:4 (geraniol: eugenol: thymol) and loaded into extracted yeast cell wall particles at a ratio of 1:1 (total terpene: YP). A schematic of this production process is shown in FIG. 17, and the resulting encapsulated di-terpene mixture is shown as YP-d(GET) EDTA.

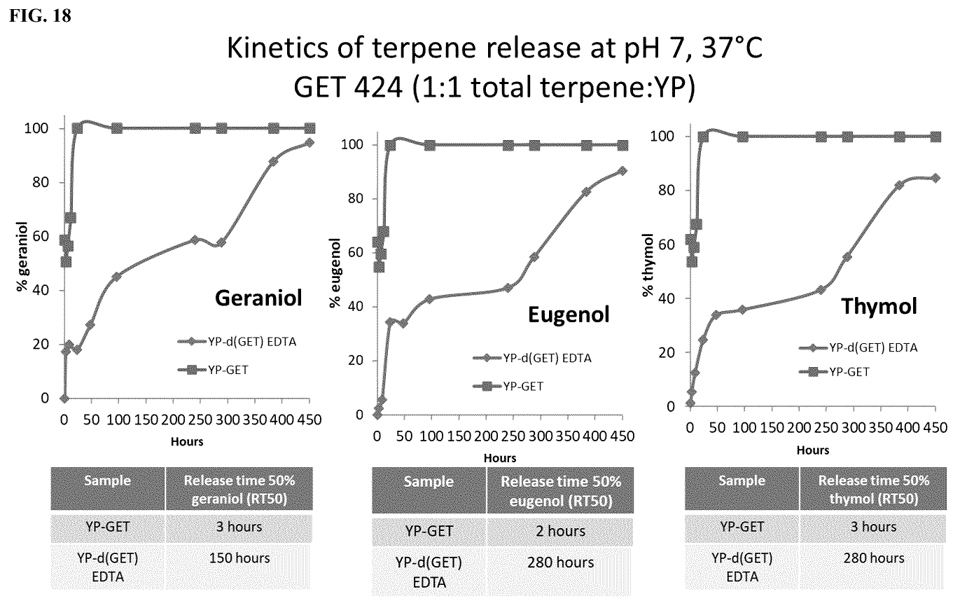

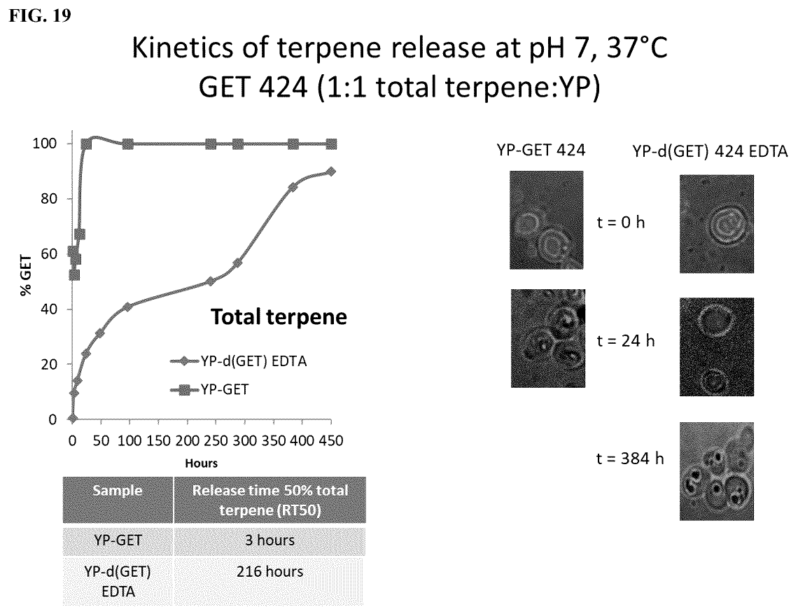

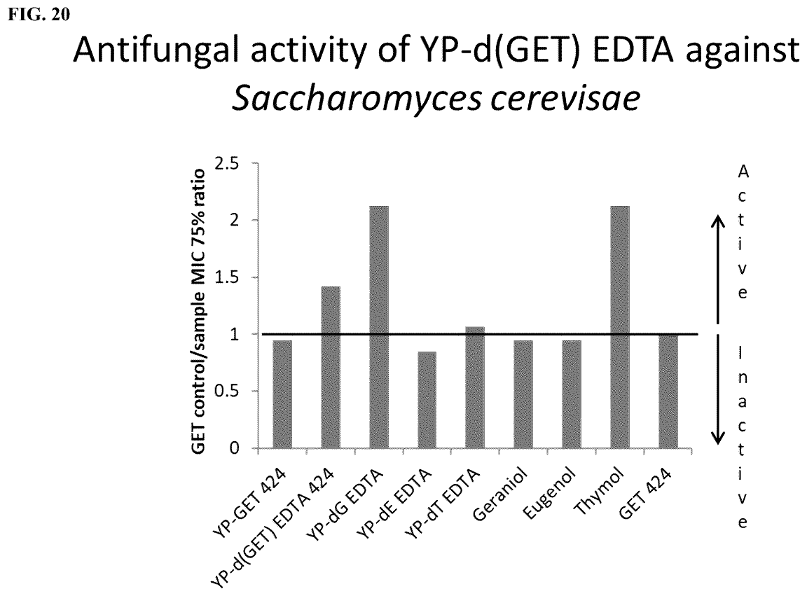

As shown in FIG. 18 and FIG. 19, encapsulated pro-payloads YP-dGeraniol EDTA, YP-dEugenol EDTA, and YP-dThymol EDTA all demonstrated vastly improved diffusion kinetics compared to a non-pro-payload mixture of geraniol, eugenol, and thymol, which had been encapsulated in yeast cell particles using conventional means (YP-GET). As shown in FIG. 20, the encapsulated mixture of terpene pro-payloads (i.e., YP-d(GET) EDTA 424), as well as the encapsulated pro-payload geraniol (i.e., YP-dG EDTA), demonstrated improved antifungal activity against the yeast S. cerevisae, compared to an unencapsulated terpene mixture or a terpene mixture encapsulated by conventional means. These data demonstrated the potential of encapsulated pro-payload terpene compounds for agricultural and medicinal uses.

### Example 4: Creation and Characterization of Pro-Payloads Comprising Carbamate or Urea Biodegradable Linkers and Payloads with Hydroxyl or Amine Groups

sA. Doxorubicin IPDI (Dox-IPDI)

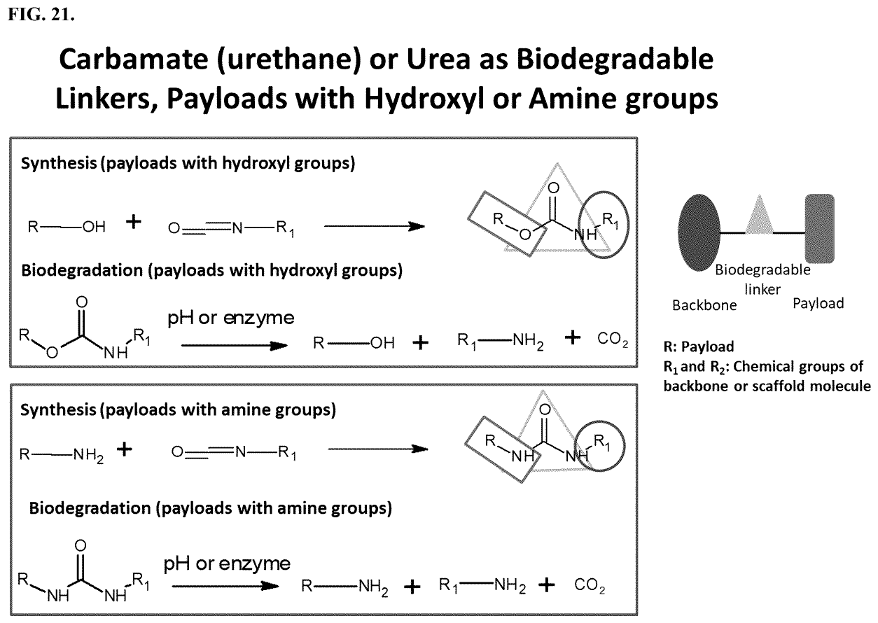

FIG. 21 shows examples of pro-payload molecules with carbamate or urea biodegradable linkers generated from payloads with hydroxyl or amine groups. The water-insoluble pro-payload doxorubicin-isophorone diisocyanate (Dox-IPDI) was synthesized by reacting doxorubicin with isophorone diisocyanate (IPDI).

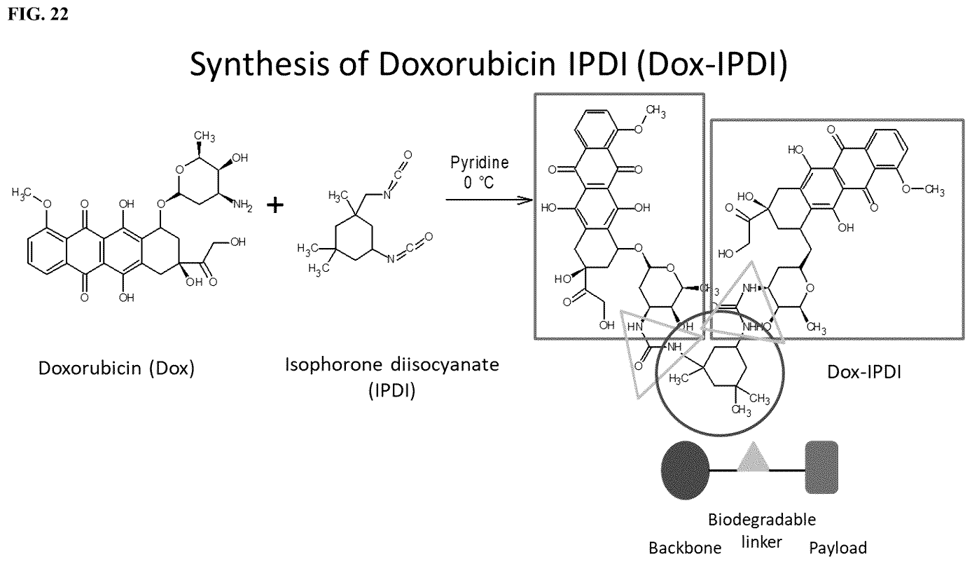

As shown in FIG. 22, pyridine catalyzed the addition of two doxorubicin payload molecules to one molecule of IPDI. The resulting urea linkages (shown schematically as yellow triangles) may be hydrolyzed by enzymatic, e.g., urease, or pH-dependent cleavage, thereby releasing the two doxorubicin payload molecules (shown schematically as green rectangles) from the IPDI backbone (shown schematically as a red circle). The pro-payload Dox-IPDI was dissolved in a suitable solvent and combined with extracted yeast cell wall particles to create encapsulated Dox-IPDI.

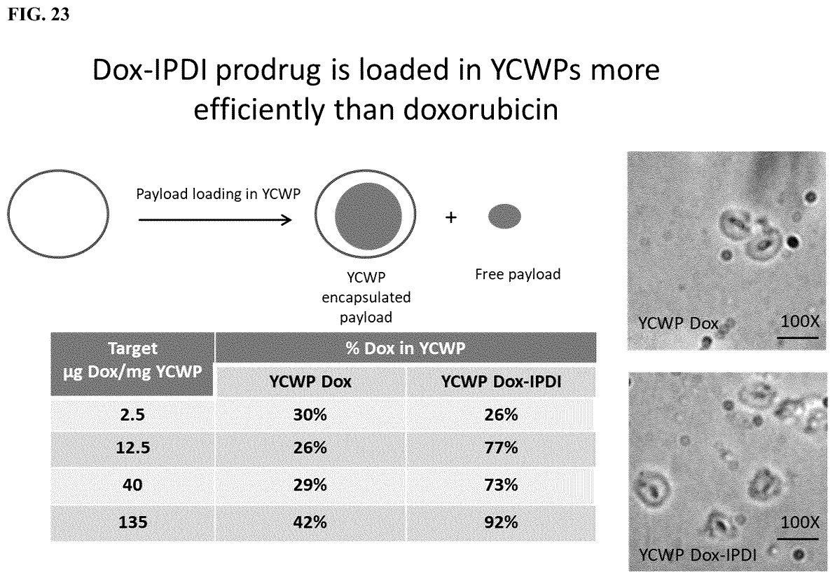

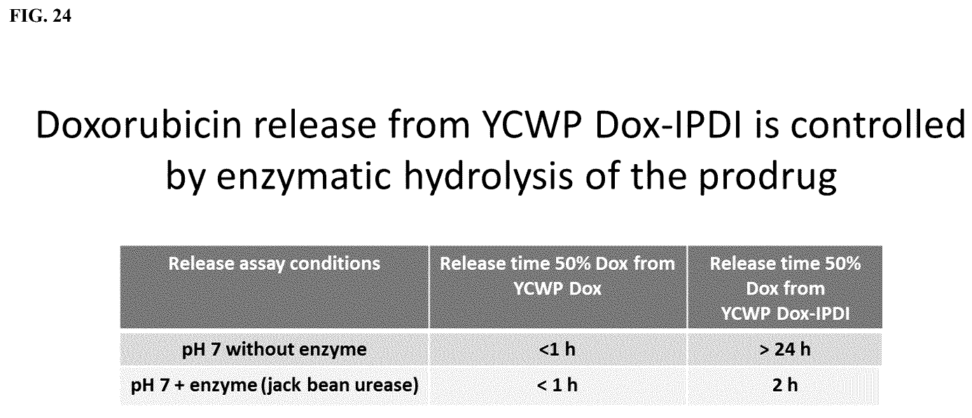

As shown in FIG. 23, considerably more Dox-IPDI could be loaded into yeast cell wall particles (e.g., up to 92%) compared to Dox alone (e.g., up to 42%), indicating that the Dox-IPDI pro-drug is loaded into YCWPs more efficiently than doxorubicin. In an in vitro release assay, YCWP-Dox-IPDI had a 50% release time (RT50) of greater than 24 hours compared to less than one (1) hour for YCWP-Dox at pH 7 in the absence of urease. In the presence of urease, Dox was readily released from YCWP-Dox-IPDI. See FIG. 24.

Glucan particles may be targeted to macrophages for delivery of encapsulated drug products. An in vitro macrophage delivery assay was performed to compare the delivery of YCWP-Dox-IPDI to YCWP-Dox and Dox alone. YCWP-Dox-IPDI, YCWP-Dox, and Dox were added to B6 macrophage cells at a ratio of 10:1, and the cells were incubated at 37° C. for approximately three (3) hours. Following a wash and an additional 12-15-hour incubation, Alamar blue dye was added to the cells. Fluorescence was measured to identify cells that remained metabolically active and able to reduce the Alamar dye.

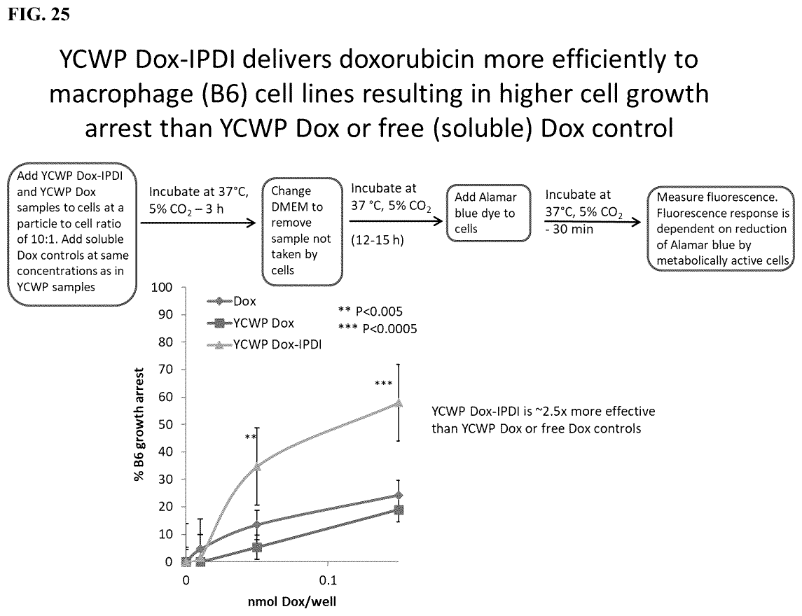

As shown in FIG. 25, YCWP-Dox-IPDI was 2.5× more effective than either YCWP-Dox or Dox at inhibiting cell growth, indicating that YCWP-Dox-IPDI delivered doxorubicin more efficiently to macrophages than either YCWP-Dox or the soluble Dox control.

### Example 5: Creation and Characterization of Pro-Payloads Comprising Hydrazone Biodegradable Linkers and Payloads with Carbonyl Groups

A. Doxorubicin Ind (dox-inh)

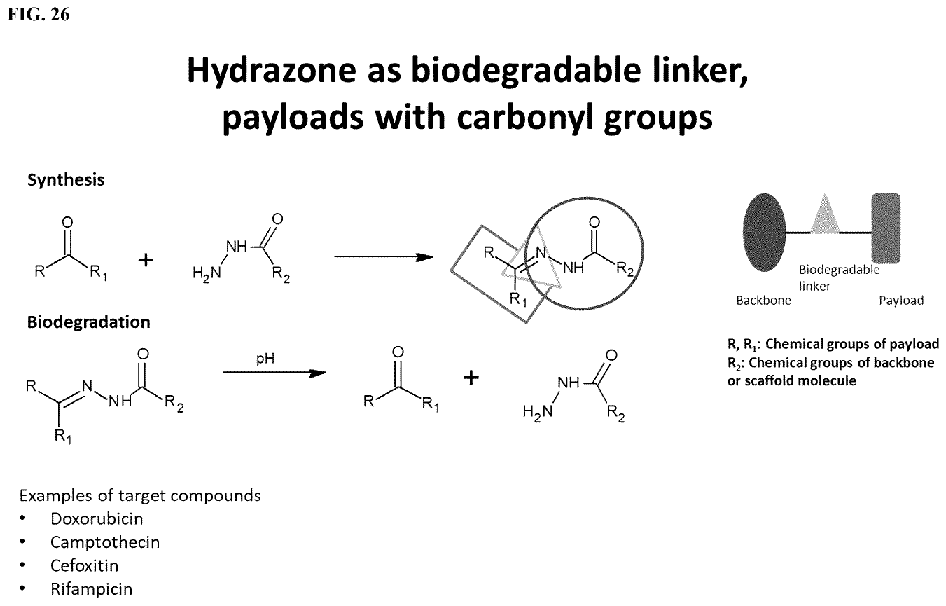

FIG. 26 shows examples of pro-payload molecules with a hydrazone biodegradable linker generated from payloads with carbonyl groups Pro-payload molecules comprising, e.g., doxorubicin, camptothecin, cefoxitin, and rifampicin, to name a few, may be generated according to the methods described.

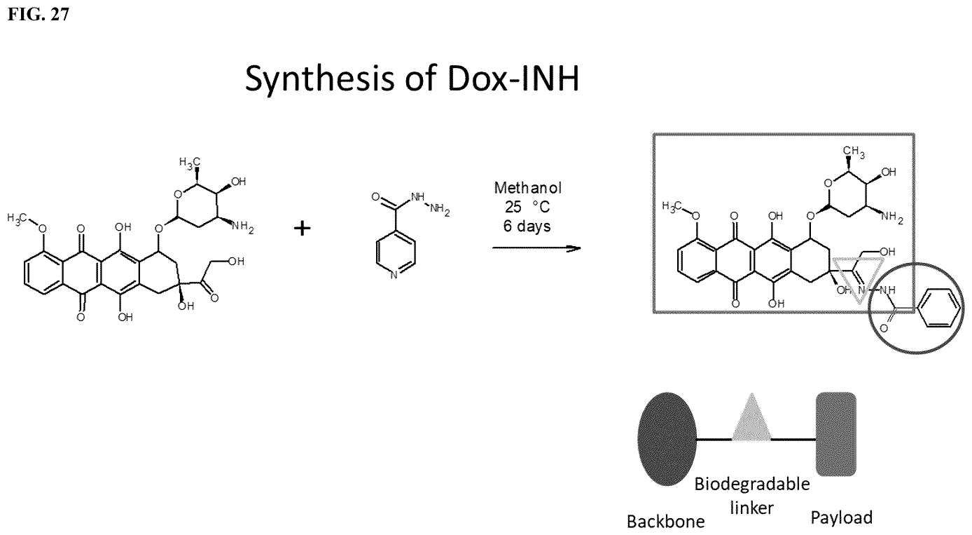

The water-insoluble pro-payload doxorubicin-isoniazid (Dox-INH) was synthesized by reacting doxorubicin with the scaffolding molecule isoniazid. As shown in FIG. 27, methanol catalyzed the addition of one doxorubicin payload molecules to one molecule of INH. The resulting hydrazone linkage (shown schematically as a yellow triangle may be hydrolyzed by pH-dependent cleavage, thereby releasing the doxorubicin payload molecule (shown schematically as a green rectangle) from the INH backbone (shown schematically as a red circle). The pro-payload Dox-INH was dissolved in a suitable solvent and combined with extracted yeast cell wall particles to create encapsulated Dox-INH.

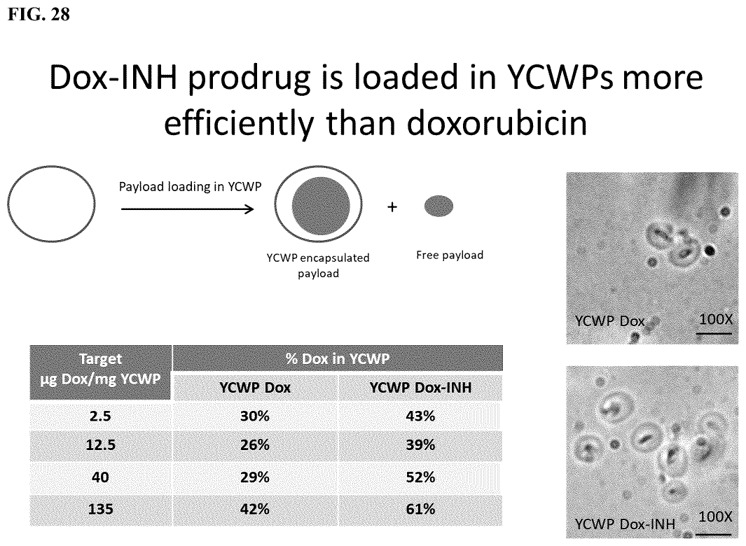

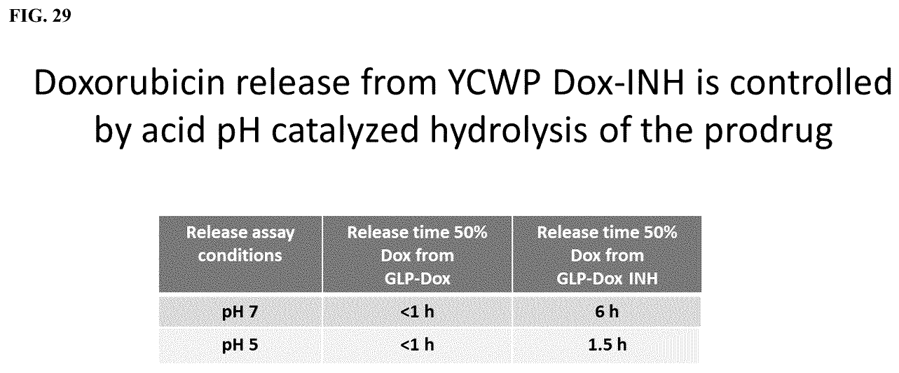

As shown in FIG. 28, considerably more Dox-INH could be loaded into yeast cell wall particles (e.g., up to 61%) compared to Dox alone (up to 42%), indicating that the Dox-INH pro-drug is loaded into YCWPs more efficiently than doxorubicin. In an in vitro release assay, YCWP-Dox-INH had a 50% release time (RT50) of six (6) hours compared to less than one (1) hour for YCWP-Dox at pH 7. At pH 5, Dox was readily released from YCWP-Dox-IPDI. See FIG. 29.

An in vitro macrophage delivery assay was performed to compare the delivery of YCWP-Dox-INH to YCWP-Dox and Dox alone. YCWP-Dox-INH, YCWP-Dox, and Dox were added to B6 macrophage cells at a ratio of 10:1, and the cells were incubated at 37° C. for approximately three (3) hours. Following a wash and an additional 12-15-hour incubation, Alamar blue dye was added to the cells. Fluorescence was measured to identify cells that remained metabolically active and able to reduce the Alamar dye.

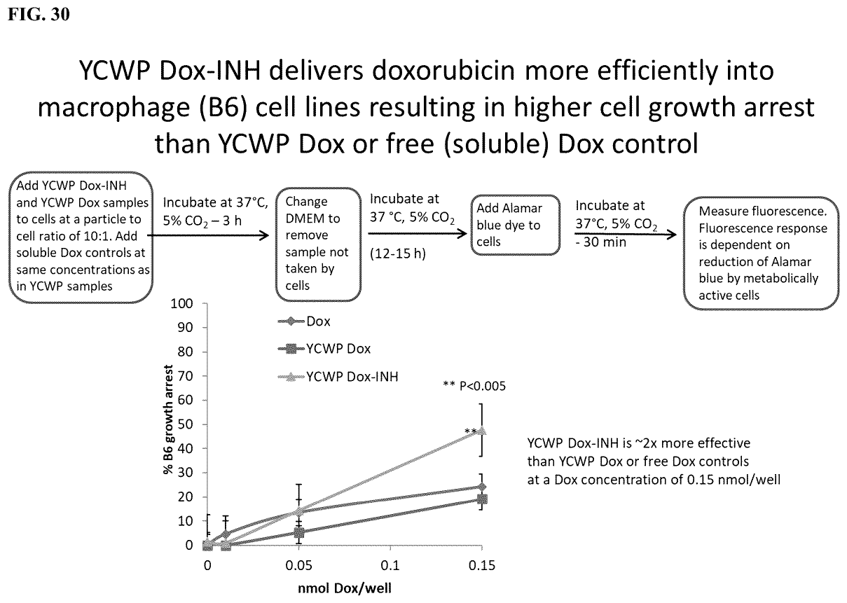

As shown in FIG. 30, YCWP-Dox-INH was 2× more effective than either YCWP-Dox or Dox at inhibiting cell growth, indicating that YCWP-Dox-INH delivered doxorubicin more efficiently to macrophages than either YCWP-Dox or the soluble Dox control.

### Example 6: Creation and Characterization of Pro-Payloads Comprising Amide Biodegradable Linkers and Payloads with Amine Groups

### A. PAMAM Doxorubicin (PAMAM-Dox) and PAMAM Cis-Aconityl Doxorubicin(PAMAM-CAD)

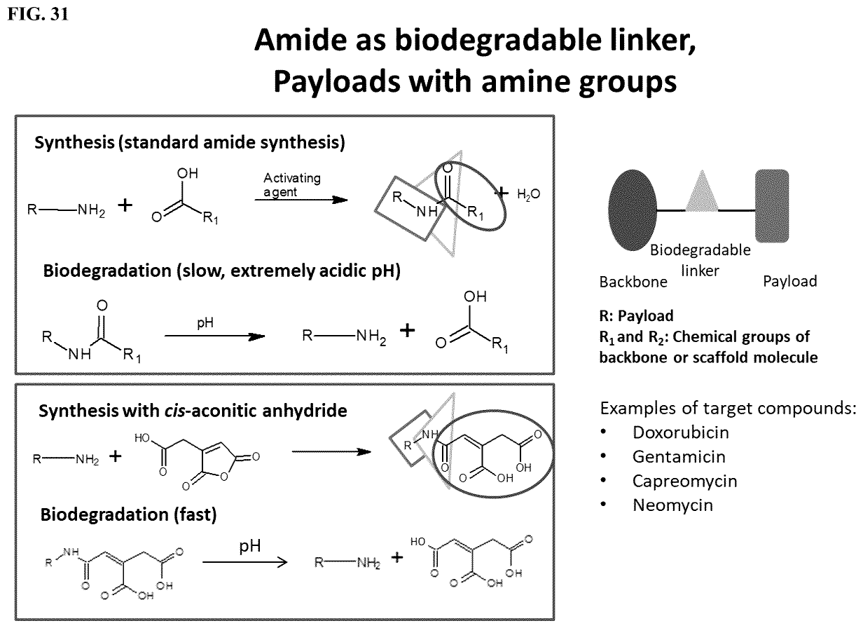

FIG. 31 shows examples of pro-payload molecules with a biodegradable amide linker generated from payloads with amine groups. Poly(amidoamine), or PAMAM, is a class of dendrimer which is made of repetitively branched subunits of amide and amine functionality. Pro-payload molecules comprising, e.g., doxorubicin, gentamicin, capreomycin, and neomycin, to name a few, may be generated according to the methods described.

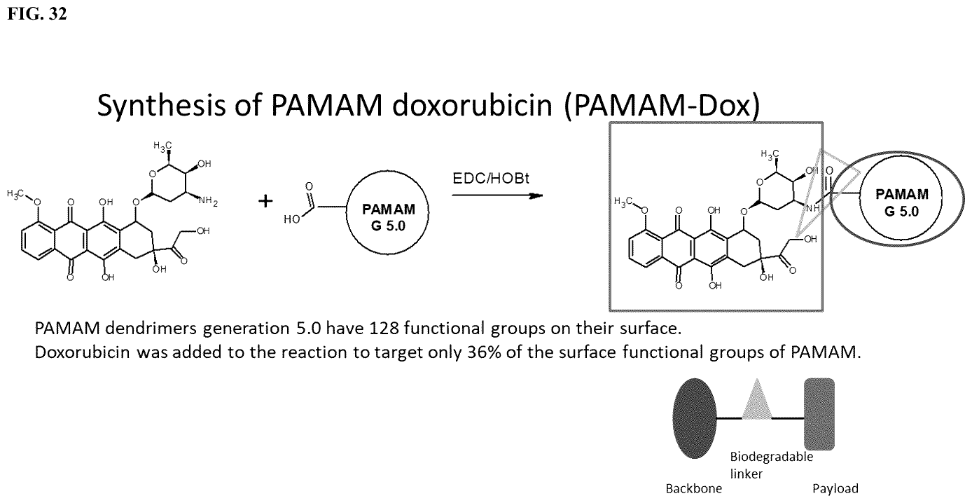

The water-insoluble pro-payload PAMAM-doxorubicin (PAMAM-Dox) was synthesized by reacting doxorubicin with PAMAM generation 5 (G 5.0), which has 128 functional groups on its surface. Doxorubicin was added to only 36% of the surface functional groups of PAMAM G 5.0. As shown in FIG. 32, the addition of doxorubicin payload molecules to one molecule of PAMAM was catalyzed using 1-ethyl-3-(3-dimethylaminopropyl)carbodiimide and hydroxybenzotriazole (EDC/HOBt). The resulting amide linkage (shown schematically as a yellow triangle) may be hydrolyzed by pH-dependent cleavage, thereby releasing the doxorubicin payload molecule (shown schematically as a green rectangle) from the PAMAM backbone (shown schematically as a red oval).

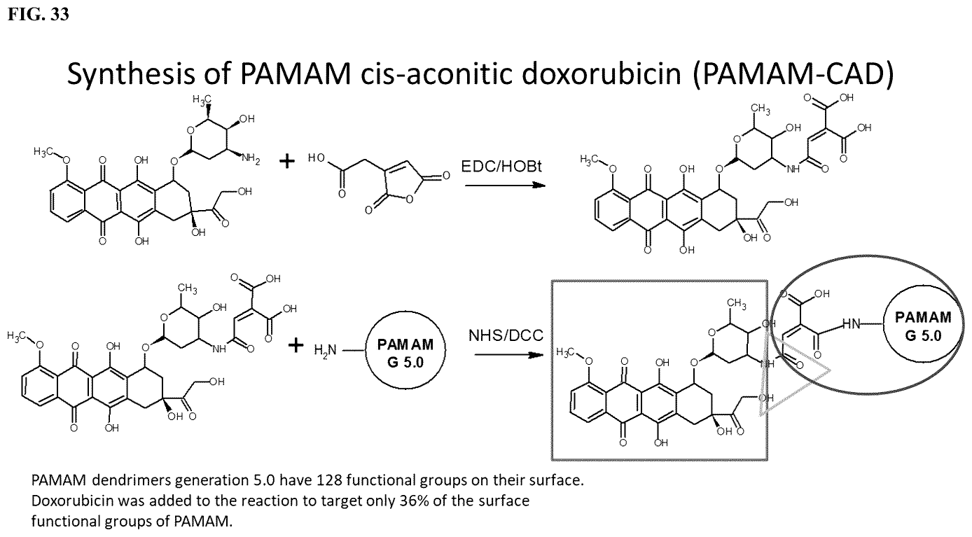

The water-insoluble pro-payload PAMAM-cis-aconitic-doxorubicin (PAMAM-CAD) was synthesized by reacting doxorubicin with the scaffolding payload molecule PAMAM and cis-aconitic anhydride. As shown in FIG. 33, the addition of one molecule of doxorubicin to one molecule of cis-Aconitic anhydride was catalyzed by (EDC/HOBt) to form cis-aconitic doxorubicin (CAD). The addition of one molecule of CAD to one molecule of PAMAM was catalyzed by N-hydroxysuccinimide and N,N′-dicyclohexylcarbodiimide (NHS/DCC) to form PAMAM-CAD. The resulting amide linkage (shown schematically as a yellow triangle) may be hydrolyzed by pH-dependent cleavage, thereby releasing the CAD payload molecule (shown schematically as a green rectangle) from the PAMAM backbone (shown schematically as a red oval). The pro-payloads PAMAM-Dox and PAMAM-CAD were dissolved in suitable solvents and combined with extracted yeast cell wall particles to create encapsulated PAMAM-Dox and PAMAM-CAD.

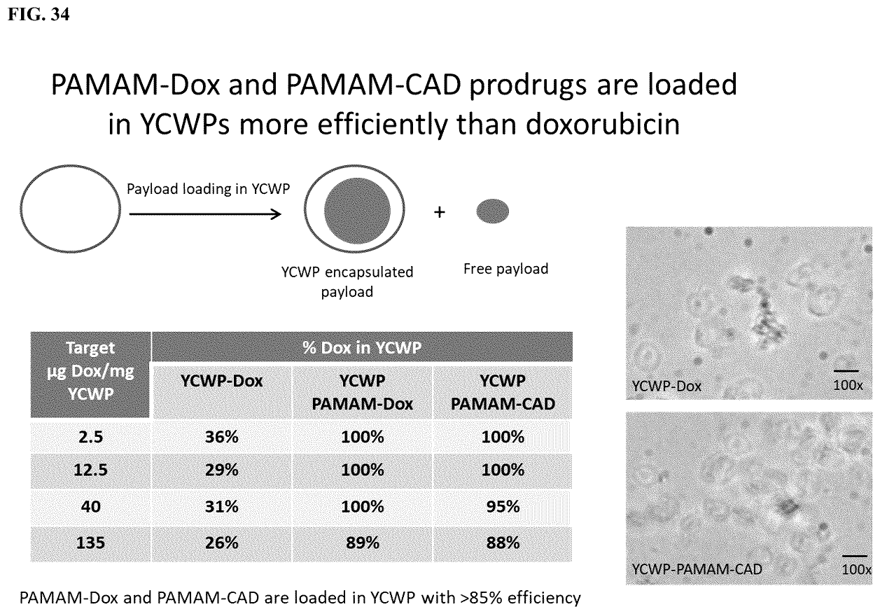

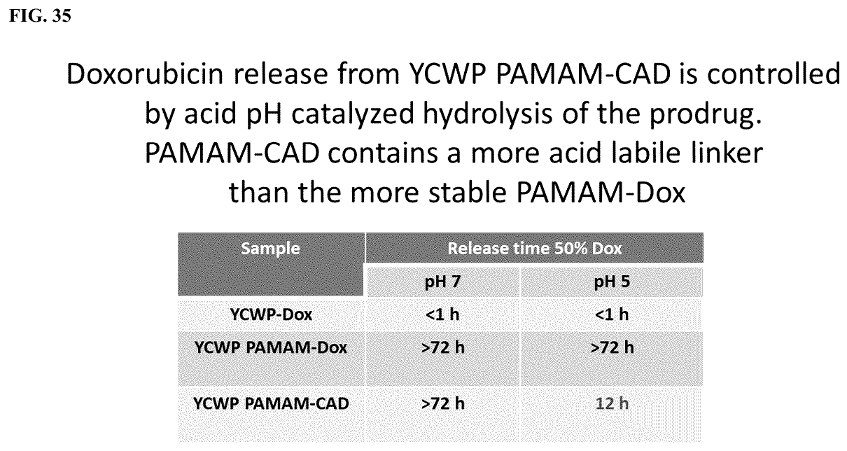

As shown in FIG. 34, considerably more PAMAM-Dox and PAMAM-CAD (i.e., up to 100%) could be loaded into yeast cell wall particles compared to Dox alone (i.e., up to 36%), indicating that the PAMAM-Dox and PAMAM-CAD pro-drugs are loaded into YCWPs more efficiently than doxorubicin. In an in vitro release assay, YCWP-PAMAM-Dox and YCWP-PAMAM-CAD had a 50% release time (RT50) of greater than 72 hours compared to less than one (1) hour for YCWP-Dox at pH 7. At pH 5, payloads were readily released from YCWP-PAMAM-CAD, whereas the payload was retained in YCWP-PAMAM-Dox, indicating that PAMAM-CAD contains a more acid labile linker than PAMAM-Dox. See FIG. 35.

An in vitro macrophage delivery assay was performed to compare the delivery of YCWP-PAMAM-Dox and YCWP-PAMAM-CAD to YCWP-Dox and Dox alone. YCWP-PAMAM-Dox, YCWP-PAMAM-CAD, YCWP-Dox, and Dox were added to B6 macrophage cells at a ratio of 10:1, and the cells were incubated at 37° C. for approximately three (3) hours. Following a wash and an additional 12-15-hour incubation, Alamar blue dye was added to the cells. Fluorescence was measured to identify cells that remained metabolically active and able to reduce the Alamar dye.

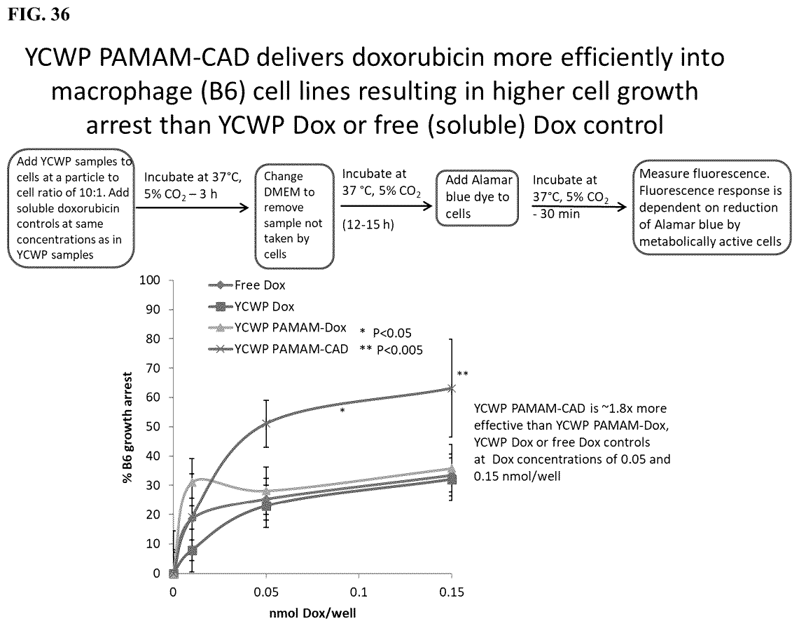

As shown in FIG. 36, YCWP-PAMAM-CAD was nearly 2× more effective than YCWP-PAMAM-Dox, YCWP-Dox, or Dox at inhibiting cell growth, indicating that YCWP-PAMAM-CAD delivered doxorubicin more efficiently to macrophages than YCWP-PAMAM-Dox, YCWP-Dox, or the soluble Dox control.

### Example 7: Creation and Characterization of Pro-Payloads Comprising Anhydride Biodegradable Linkers and Payloads with Hydroxyl or Carboxylic Acid Groups

### A. Naproxen Anhydride (Nap-An)

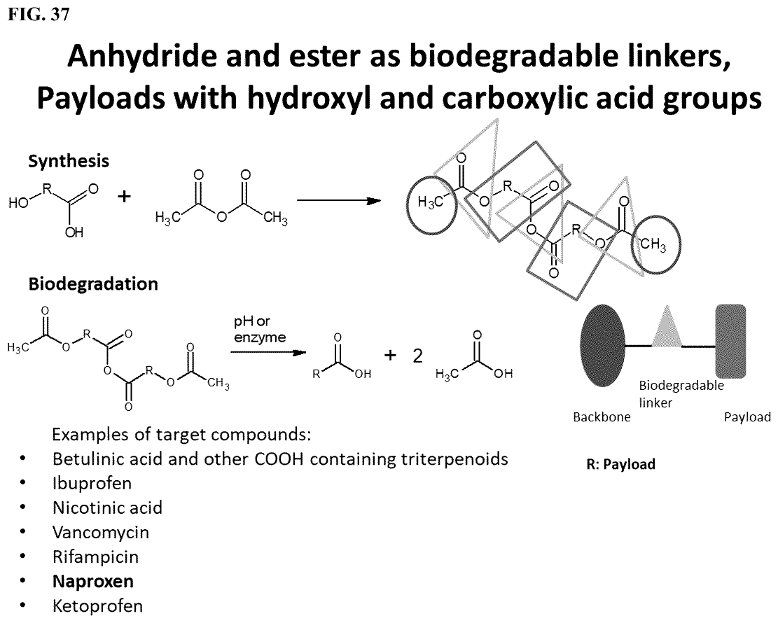

FIG. 37 shows examples of pro-payload molecules with a biodegradable ester linker generated from payloads with hydroxyl and/or carboxylic acid groups. Pro-payload molecules comprising, e.g., ibuprofen, nicotinic acid, vancomycin, rifamycin, naproxen, ketoprofen, and betulinic acid, as well as other COOH-containing triterpenoids, to name a few, may be generated according to the methods described.

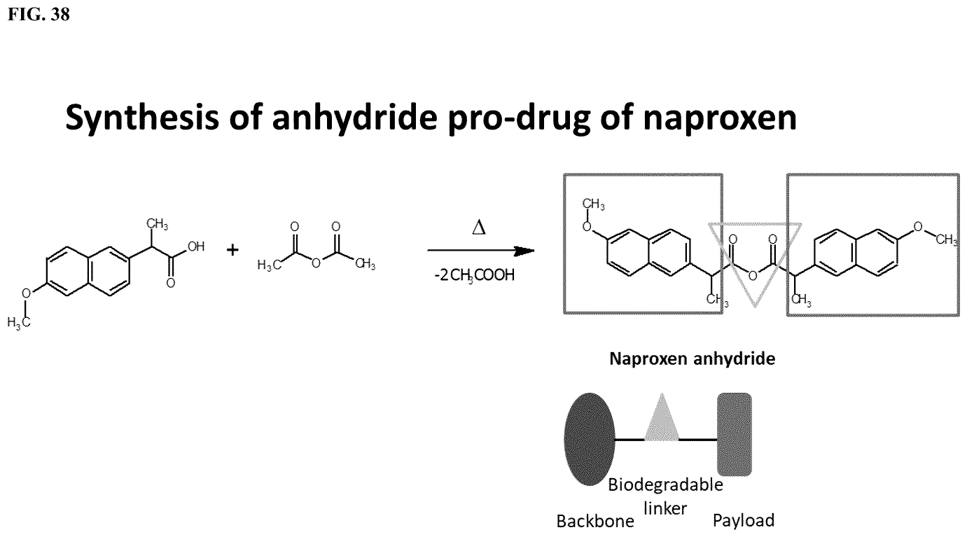

The water-insoluble pro-payload naproxen anhydride (Nap-An) was synthesized by reacting naproxen with ethanoic anhydride (CH₃CO)₂O. As shown in FIG. 38, acetic acid catalyzed the reaction of two molecules of naproxen with ethanoic anhydride. The resulting carboxylic ester linkage (shown schematically as a yellow triangle) may be hydrolyzed by pH-dependent cleavage, thereby releasing the naproxen payload molecule (shown schematically as green rectangles).

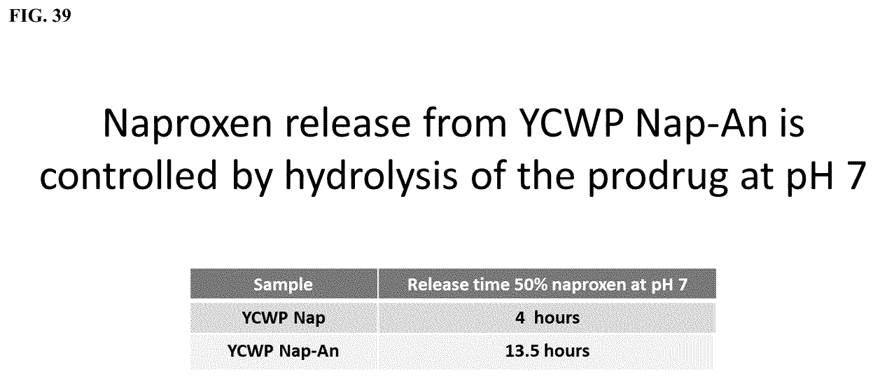

The pro-payload Nap-An was dissolved in a suitable solvent and combined with extracted yeast cell wall particles to create encapsulated Nap-An. In an in vitro release assay, Nap-An had a 50% release time (RT50) of greater than 13.5 hours compared to less than one (1) hour for YCWP-Nap at pH 7. See FIG. 39.

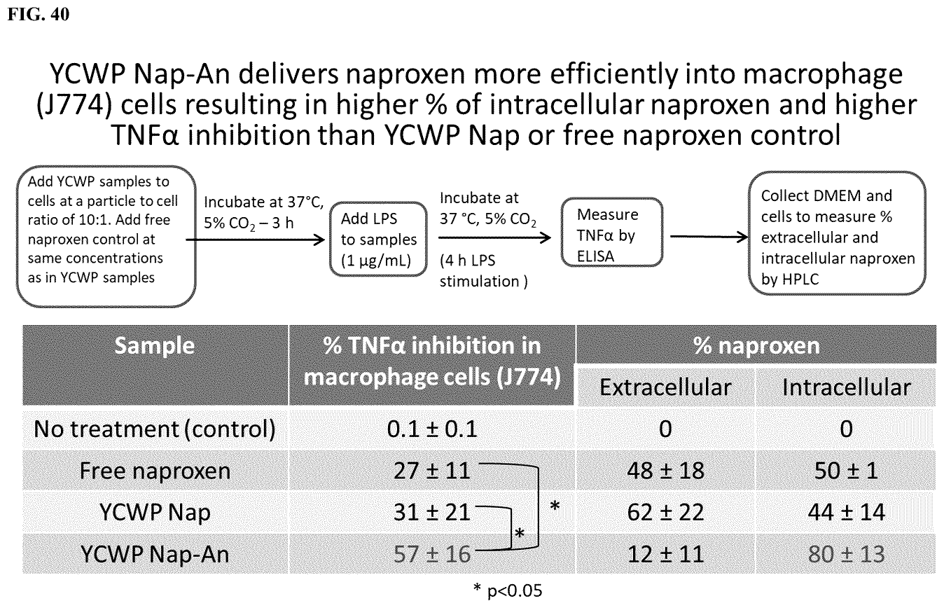

An in vitro macrophage delivery assay was performed to compare the delivery of YCWP-Nap-An to YCWP-Nap and Nap alone. YCWP-Nap-An, YCWP-Nap, and Nap were added to J 774 macrophage cells at a ratio of 10:1, and the cells were incubated at 37° C. for approximately three (3) hours. Following the addition of lipopolysaccharide (LPS) and an additional 4-hour incubation, TNF-alpha was measured by ELISA and the percentage of extracellular and intracellular naproxen was measured by HPLC. As shown in FIG. 40, YCWP-Nap-An was delivered intracellularly to macrophages nearly 2× more effectively than YCWP-Nap. In addition, YCWP-Nap-An resulted in significantly greater inhibition of TNF-alpha in macrophages compared to either YCWP-Nap or the free Nap control.

### Example 8: Creation and Characterization of Pro-Payloads Comprising Enamine Biodegradable Linkers and Payloads with Amine, Ketone, or Aldehyde Groups

### A. Cycloserine Acetylacetone (CS-AcA)

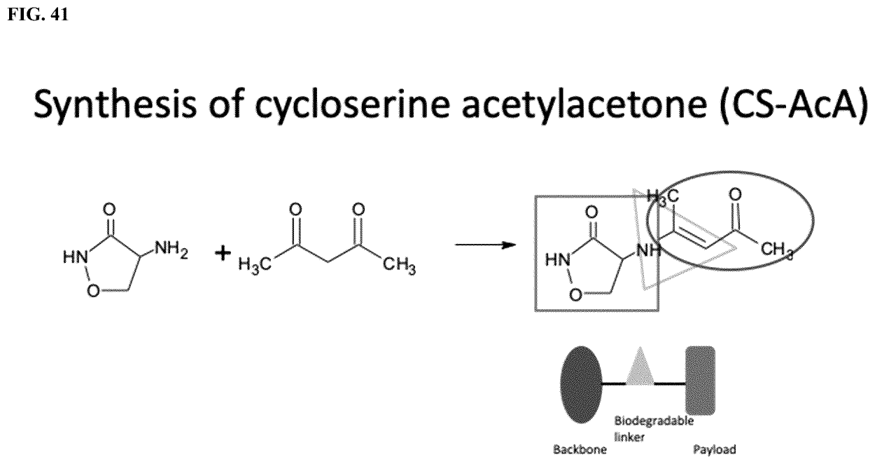

FIG. 41 shows an example of a pro-payload molecule with a biodegradable enamine linker generated from payloads with amine, ketone, or aldehyde groups. The sparingly water-soluble pro-payload cycloserine acetylacetone (CS-AcA) was synthesized by reacting cycloserine with the scaffolding payload molecule acetyleacetone. The resulting enamine linkage (shown schematically as a yellow triangle) may be hydrolyzed, thereby releasing the CS payload molecule (shown schematically as a green rectangle) from the AcA backbone (shown schematically as a red oval).

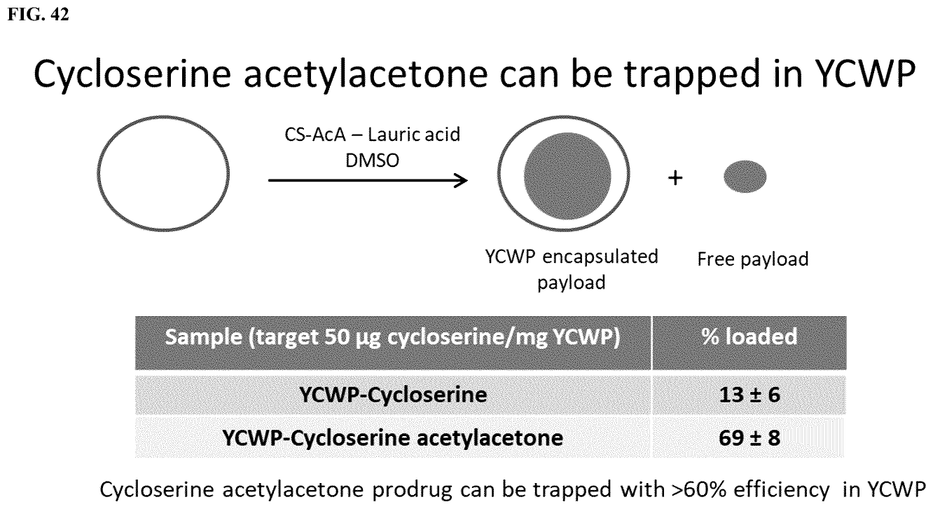

The pro-payload CS-AcA was dissolved in a suitable solvent and combined with extracted yeast cell wall particles to create encapsulated CS-AcA. As shown in FIG. 42, considerably more CS-AcA (i.e., up to approximately 70%) could be loaded into yeast cell wall particles compared to CS alone (i.e., up to approximately 14%), indicating that the CS-AcA pro-drugs are loaded into YCWPs more efficiently than CS alone.

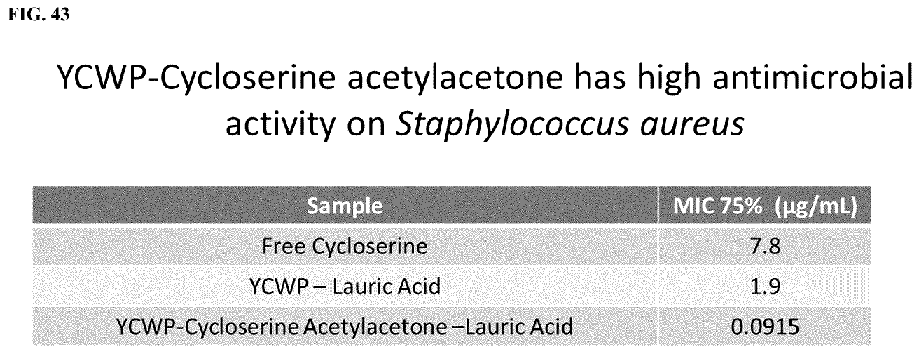

An in vitro antimicrobial assay was performed to determine whether YCWP-CS-AcA retained the ability to inhibit the survival of the bacterium Staphylococcus aureus. As shown in FIG. 43, YCWP-CS-AcA was highly antimicrobial compared to either YCWP-LA (lauric acid) or free cycloserine.

### Example 9: Creation and Characterization of Pro-Payloads Comprising Hydrazide Biodegradable Linkers and Payloads with Amine, Hydrazine, or Carbonyl Groups

### A. Isoniazid-Lauric Acid (INH-LA)

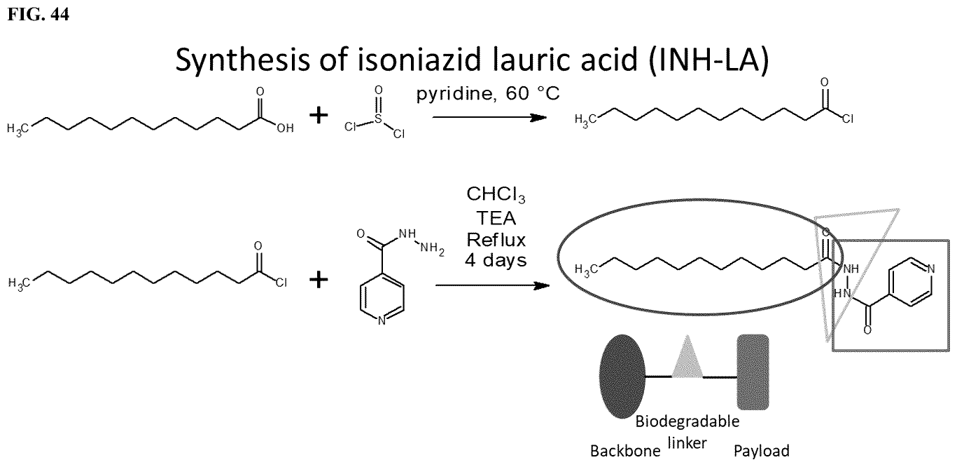

FIG. 44 shows an example of a pro-payload molecule with a biodegradable hydrazide linker generated from payloads with carboxylic acid groups. The water-insoluble pro-payload isoniazid lauric acid (INH-LA) was synthesized by: a) reacting lauric acid (LA) with a halogenating agent, e.g., thionyl chloride, to form the acid halide; and b) reacting isoniazid (INH) with lauric acid halide, e.g., lauric acid chloride, to form the pro-payload isoniazid lauric acid. The resulting hydrazide linkage (shown schematically as a yellow triangle) may be hydrolyzed, thereby releasing the INH payload molecule (shown schematically as a green rectangle) from the LA backbone (shown schematically as a red oval).

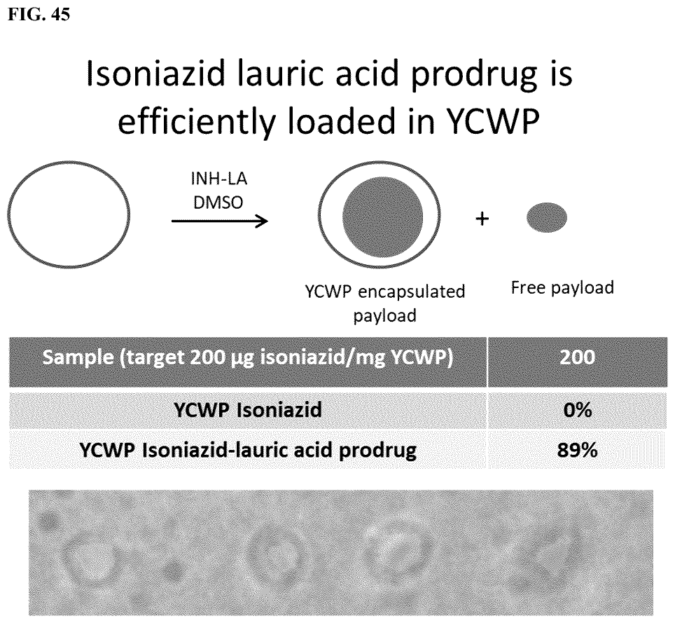

The pro-payload INH-LA was dissolved in a suitable solvent and combined with extracted yeast cell wall particles to create encapsulated INH-LA. As shown in FIG. 45, considerably more INH-LA (i.e., up to 89%) could be loaded into yeast cell wall particles compared to INH alone (i.e., 0%), indicating that the INH-LA pro-drugs are loaded into YCWPs much more efficiently than INH alone.

The following claims should not be read as limited to the described order or elements unless stated to that effect. Therefore, all embodiments that come within the scope and spirit of the following claims and equivalents thereto are claimed as the disclosure.

1-36. (canceled)

37. A method of delivering a payload molecule to a subject, comprising:
(a) linking a payload molecule to a scaffolding molecule via a cleavable linker to form a water-insoluble pro-payload molecule;
(b) contacting an extracted yeast cell wall particle (YCWP) with the water-insoluble pro-payload molecule, wherein the extracted YCWP comprises an internal space and beta glucan, and wherein the water-insoluble pro-payload molecule is at least partially enclosed within the internal space, thereby forming a particulate delivery system; and
(c) administering to a subject the particulate delivery system under conditions that permit internalization of the particulate delivery system in a cell of the subject, cleavage of the cleavable linker, and release and delivery of the payload molecule within the cell o f the subject.

38. The method of claim 37, wherein the linker is selected from the group consisting of an amide, an acetal, an anhydride, an aminocarboxylic acid, a carbamate, a cycloalkane, a disulfide, an enamine, an ester, a polyester, a hydrazide, a hydrazone, and urea.

39. The method of claim 37, wherein the linker is cleavable by chemical or enzymatic hydrolysis, optionally wherein the linker is cleavable by pH-dependent chemical hydrolysis.

40. (canceled)

41. The method of claim 39, wherein the linker is cleavable with a reagent selected from the group consisting of an enzyme, a reducing agent, an oxidizing agent, an acid, a base, and an organometallic or metal reagent,
optionally wherein the enzyme is selected from the group consisting of a carboxylase, an esterase, and a urease.

42. (canceled)

43. The method of claim 37, wherein the payload scaffolding molecule comprises a chemical moiety selected from the group consisting of acetylacetone, anhydride, cyclohexane, cyclohexane 1,2,4, 5, tetracarboxylic acid, ethylenediaminetetraacetic acid (EDTA), isophorone diisocyanate, lauric acid, and poly(amidoamine).

44. The method of claim 37, wherein the extracted yeast cell wall further comprises less than 90 weight percent beta-glucan,
optionally wherein the extracted yeast cell wall further comprises less than 30 weight percent chitin.

45. (canceled)

46. The method of claim 37, wherein the payload molecule comprises a reactive moiety selected from the group consisting of an amine, an aldehyde, a carbonyl, a carboxylic acid, a hydrazine, a hydroxyl, and a ketone,
optionally wherein the reactive moiety is a hydroxyl.

47. The method of claim 37, wherein the payload molecule is selected from the group consisting of a polynucleotide, a peptide, a protein, a small organic active agent, a small inorganic active agent, a microbicide, a fungicide, an insecticide, a nematocide, a pesticide, an antibiotic, an analgesic, a non-steroidal anti-inflammatory drug (NSAID), a terpene, a terpenoid, a chemotherapeutic, and a dietary supplement, and any mixtures thereof,
optionally wherein the payload molecule is selected from the group consisting of carvacrol, eugenol, geraniol, resveratrol, acetaminophen, and curcumin.

48. (canceled)

49. (canceled)

50. The method of claim 47, wherein the payload molecule is carvacrol, and the pro-payload molecule is dicarvacrol-EDTA.

51. The method of claim 47, wherein the payload molecule is carvacrol, and the pro-payload molecule is dicarvacrol-cyclohexane.

52. The method of claim 47, wherein the payload molecule is selected from the group consisting of geraniol, eugenol, and thymol, or a combination thereof, and the pro-payload molecule is selected from the group consisting of di-geraniol-EDTA, di-eugenol-EDTA, and di-thymol-EDTA, or a combination thereof.

53. The method of claim 46, wherein the reactive moiety is an amine,
optionally wherein the payload molecule is selected from the group consisting of daunomycin, doxorubicin, cis-aconityl-doxorubicin, gentamicin, capreomycin, neomycin, and acetaminophen.

54. (canceled)

55. The method of claim 53, wherein the payload molecule is doxorubicin, and the pro-payload molecule is poly(amidoamine)-doxorubicin.

56. The method of claim 53, wherein the payload molecule is cis-aconityl-doxorubicin, and the pro-payload molecule is poly(amidoamine)-cis-aconityl-doxorubicin.

57. The method of claim 46, wherein the reactive moiety is a carbonyl
optionally wherein the payload molecule is selected from the group consisting of doxorubicin, gentamicin, neomycin, cefoxitin, rifampicin, and camptothecin.

58. (canceled)

59. The method of claim 57, wherein the payload molecule is doxorubicin, and the pro-payload molecule is doxorubicin-isoniazid.

60. The method of claim 46, wherein the reactive moiety is a carboxylic acid or a hydroxyl,
optionally wherein the payload molecule is selected from the group consisting of ibuprofen, nicotinic acid, vancomycin, rifampicin, naproxen, ketoprofen, and betulinic acid or other carboxylic acid containing triterpenoids.

61. (canceled)

62. The method of claim 60, wherein the payload molecule is naproxen, and the pro-payload molecule is naproxen-anhydride.

63. The method of claim 46, wherein the reactive moiety is selected from the group consisting of an amine and a hydroxyl, optionally wherein the payload molecule is selected from the group consisting of carvacrol and doxorubicin.

64. (canceled)

65. The method of claim 63, wherein the payload molecule is doxorubicin, and the pro-payload molecule is doxorubicin-isophorone diisocyanate.

66. The method of claim 46, wherein the reactive moiety is selected from the group consisting of an amine, a ketone, and an aldehyde.

67. The method of claim 66, wherein the payload molecule is cycloserine, and the pro-payload molecule is cycloserine-acetylacetone.

68. The method of claim 46, wherein the reactive moiety is selected from the group consisting of an amine, a hydrazine, and a carbonyl.

69. The method of claim 68, wherein the payload molecule is isoniazid, and the pro-payload molecule is isoniazid-lauric acid.

70-102. (canceled)

103. A method of delivering a payload molecule to a subject, comprising:
(a) linking a payload molecule to a scaffolding molecule via a cleavable linker to form a water-insoluble pro-payload molecule;
(b) contacting an extracted yeast cell wall particle (YCWP) with the water-insoluble pro-payload molecule, wherein the extracted YCWP comprises an internal space and beta glucan, and wherein the water-insoluble pro-payload molecule is at least partially enclosed within the internal space, thereby forming a particulate delivery system; and
(c) administering the particulate delivery system to a subject under conditions that permit release and delivery of the payload molecule in the subject.

104. The method of claim 103, wherein the particulate delivery system is delivered to the subject by oral, buccal, sublingual, pulmonary, or transmucosal administration.

105. The method of claim 103, wherein the particulate delivery system is delivered to the subject as a pharmaceutically acceptable composition.

106. The method of claim 105, wherein the pharmaceutically acceptable composition is administered to the subject topically, orally, rectally, vaginally or via a pulmonary route.

107. A method of delivering a payload molecule to a subject, comprising:
(a) linking a payload molecule to a scaffolding molecule via a cleavable linker to form a water-insoluble pro-payload molecule;
(b) contacting an extracted yeast cell wall particle (YCWP) with the water-insoluble pro-payload molecule, wherein the extracted YCWP comprises an internal space and beta glucan, and wherein the water-insoluble pro-payload molecule is at least partially enclosed within the internal space, thereby forming a particulate delivery system;
(c) formulating the particulate delivery system as a pharmaceutically acceptable composition; and
(c) administering the pharmaceutically acceptable composition to a subject under conditions that permit release and delivery of the payload molecule in the subject.
